SEQUENCE LISTING (80 sequences)

SEQ ID NO: 1 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 2 - protein, 15 aa, Homo sapiens
NVASSTNLLTDSKSC

SEQ ID NO: 3 - protein, 12 aa, Homo sapiens
SAGVTHSVARSC

SEQ ID NO: 4 - protein, 13 aa, Homo sapiens
HNNEAGRTTVFSC

SEQ ID NO: 5 - protein, 14 aa, Homo sapiens
LEPEDPAHKPPSPC

SEQ ID NO: 6 - protein, 23 aa, Homo sapiens
DGCELLCCGRGHRTRTQRVTERC

SEQ ID NO: 7 - protein, 17 aa, Homo sapiens
HVSCRNCTHTRVLHECL

SEQ ID NO: 8 - protein, 360 aa, Homo sapiens
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 9 - protein, 15 aa, Homo sapiens
SSQRQLCHRHPDVMR

SEQ ID NO: 10 - protein, 14 aa, Homo sapiens
CDPKKMGSAKDSKG

SEQ ID NO: 11 - protein, 13 aa, Homo sapiens
VDAKERKGKDARC

SEQ ID NO: 12 - protein, 18 aa, Homo sapiens [sequence omitted]

SEQ ID NO: 13 - protein, 355 aa, Homo sapiens
MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 14 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 15 - protein, 351 aa, Homo sapiens
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 16 - protein, 365 aa, Homo sapiens
MAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 17 - protein, 359 aa, Homo sapiens
MPSLLLLFTAALLSSWAQLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 18 - protein, 365 aa, Homo sapiens
MLPPLPSRLGLLLLLLLCPAHVGGLWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 19 - protein, 349 aa, Homo sapiens
MNRKALRCLGHLFLSLGMVCLRIGGFSSVVALGATIICNKIPGLAPRQRAICQSRPDAIIVIGEGSQMGLDECQFQFRNGRWNCSALGERTVFGKELKVGSRDGAFTYAIIAAGVAHAITAACTHGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 20 - protein, 349 aa, Homo sapiens
MHRNFRKWIFYVFLCFGVLYVKLGALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 21 - protein, 355 aa, Homo sapiens
MGNLFMLWAALGICCAAFSASAWSVNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGRVWFGVYI

SEQ ID NO: 22 - protein, 351 aa, Homo sapiens
MFLSKPSVYICLFTCVLQLSHSWSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAARGAIADTFRSISTRELVHLEDSPDYCLENKTLGLLGTEGRECLRRGRALGRWELRSCRRLCGDCGLAVEERRAETVSSCNCKFHWCCAVRCEQCRRRVTKYFCSRAERPRGGAAHKPGRKP

SEQ ID NO: 23 - protein, 417 aa, Homo sapiens
MGSAHPRPWLRLRPQPQPRPALWVLLFFLLLLAAAMPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 24 - protein, 389 aa, Homo sapiens
MLEEPRPRPPPSGLAGLLFLALCSRALSNEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 25 - protein, 354 aa, Homo sapiens [sequence omitted]

SEQ ID NO: 26 - protein, 389 aa, Homo sapiens; identical to SEQ ID NO: 24

SEQ ID NO: 27 - protein, 391 aa, Homo sapiens
MLRPGGAEEAAQLPLRRASAPVPVPSPAAPDGSRASARLGLACLLLLLLLTLPARVDTSWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 28 - protein, 365 aa, Homo sapiens
MLDGSPLARWLAAAFGLTLLLAALRPSAAYFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 29 - protein, 357 aa, Homo sapiens [sequence omitted]

SEQ ID NO: 30 - protein, 361 aa, Homo sapiens [sequence omitted]

SEQ ID NO: 31 - protein, 318 aa, Homo sapiens
MAEEEAPKKSRAAGGGASWELCAGALSARLAEEGSGDAGGRRRPPVDPRRLARQLLLLLWLLEAPLLLGVRAQAAGQGPGQGPGPGQQPPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGHRGGFPGGAGASERGKFSCPRALKVPSYLNYHFLGEKDCGAPCEPTKVYGLMYFGPEELR

SEQ ID NO: 32 - protein, 242 aa, Homo sapiens
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETR

SEQ ID NO: 33 - protein, 200 aa, Homo sapiens
MAMTWIVFSLWPLTVFMGHIGGHSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDLNLAGEPTEGAPVAVQRDYGFWCPRELKIDPDLGYSFLHVRDCSPPCPNMYFRREELS

SEQ ID NO: 34 - protein, 217 aa, Homo sapiens
MAWRGAGPSVPGAPGGVGLSLGLLLQLLLLLGPARGFGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEVPLPHKTPIQPGEECHSVGTNSDQYIWVKRSLNCVLKCGYDAGLYSRSAKE

SEQ ID NO: 35 - protein, 233 aa, Homo sapiens
MARPDPSAPPSLLLLLLAQLVGRAAAASKAPVCQEITVPMCRGIGYNLTHMPNQFNHDTQDEAGLEVHQFWPLVEIQCSPDLRFFLCTMYTPICLPDYHKPLPPCRSVCERAKAGCSPLMRQYGFAWPERMSCDRLPVLGRDAEVLCMDYNRSEATTAPPRPFPAKPTLPGPPGAPASGGECPAGGPFVCKCREPFVPILKESHPLYNKVRTGQVPNCAVPCYQPSFSADERT

SEQ ID NO: 36 - protein, 196 aa, Homo sapiens
MEMFTFLLTCIFLPLLRGHSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLGPQKKTEQVQRDIGFWCPRHLKTSGGQGYKFLGIDQCAPPCPNMYFKSDELE

SEQ ID NO: 37 - protein, 251 aa, Homo sapiens
MRDPGAAVPLSSLGFCALVLALLGALSAGAGAQPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGPGGGPTAYPTAPYLPDLPFTALPPGASDGKGRPAFPFSCPRQLKVPPYLGYRFLGERDCGAPCEPGRANGLMYFKEEERR

SEQ ID NO: 38 - protein, 275 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERA

SEQ ID NO: 39 - protein, 229 aa, Homo sapiens
MAVAPLRGALLLWQLLAAGGAALEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENATAGPAEPHKGLGMLPVAPRPARPPGDLGPGAGGSGTCENPEKFQYVEKSRSCAPRCGPGVEVFWSRRDKD

SEQ ID NO: 40 - protein, 225 aa, Homo sapiens
MQRPGPRLWLVLQVMGSCAAISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNGSDEPTRGSGLFPPLFRPQRPHSAQEHPLKDGGPGRGGCDNPGKFHHVEKSASCAPLCTPGVDVYWSREDKR

SEQ ID NO: 41 - protein, 716 aa, Homo sapiens
MGETKIIYHLDGQETPYLVKLPLPAERVTLADFKGVLQRPSYKFFFKSMDDDFGVVKEEISDDNAKLPCFNGRVVYWLVSAEGSHPDPAPFCADNPSELPPPMERTGGIGDSRPPSFHPHAGGGSQENLDNDTETDSLVSAQRERPRRRDGPEHATRLNGTAKGERRREPGGYDSSSTLMSSELETTSFFDSDEDDSTSRFSSSTEQSSASRLMRRHKRRRRKQKVSRIERSSSFSSITDSTMSLNIITVTLNMEKYNFLGISIVGQSNERGDGGIYIGSIMKGGAVAADGRIEPGDMLLQVNEINFENMSNDDAVRVLREIVHKPGPITLTVAKCWDPSPRGCFTLPRSEPIRPIDPAAWVSHTAAMTGTFPAYGMSPSLSTITSTSSSITSSIPDTERLDDFHLSIHSDMAAIVKAMASPESGLEVRDRMWLKITIPNAFIGSDVVDWLYHNVEGFTDRREARKYASNLLKAGFIRHTVNKITFSEQCYYIFGDLCGNMANLSLHDHDGSSGASDQDTLAPLPHPGAAPWPMAFPYQYPPPPHPYNPHPGFPELGYSYGGGSASSQHSEGSRSSGSNRSGSDRRKEKDPKAGDSKSGGSGSESDHTTRSSLRGPRERAPSERSGPAASEHSHRSHHSLASSLRSHHTHPSYGPPGVPPLYGPPMLMMPPPPAAMGPPGAPPGRDLASVPPELTASRQSFRMAMGNPSEFFVDVM

SEQ ID NO: 42 - protein, 670 aa, Homo sapiens
MAETKIIYHMDEEETPYLVKLPVAPERVTLADFKNVLSNRPVHAYKFFFKSMDQDFGVVKEEIFDDNAKLPCFNGRVVSWLVLAEGAHSDAGSQGTDSHTDLPPPLERTGGIGDSRPPSFHPNVASSRDGMDNETGTESMVSHRRERARRRNREEAARTNGHPRGDRRRDVGLPPDSASTALSSELESSSFVDSDEDGSTSRLSSSTEQSTSSRLIRKHKRRRRKQRLRQADRASSFSSITDSTMSLNIVTVTLNMERHHFLGISIVGQSNDRGDGGIYIGSIMKGGAVAADGRIEPGDMLLQVNDVNFENMSNDDAVRVLREIVSQTGPISLTVAKCWDPTPRSYFTVPRADPVRPIDPAAWLSHTAALTGALPRYELEEAPLTVKSDMSAVVRVMQLPDSGLEIRDRMWLKITIANAVIGADVVDWLYTHVEGFKERREARKYASSLLKHGFLRHTVNKITFSEQCYYVFGDLCSNLATLNLNSGSSGTSDQDTLAPLPHPAAPWPLGQGYPYQYPGPPPCFPPAYQDPGFSYGSGSTGSQQSEGSKSSGSTRSSRRAPGREKERRAAGAGGSGSESDHTAPSGVGSSWRERPAGQLSRGSSPRSQASATAPGLPPPHPTTKAYTVVGGPPGGPPVRELAAVPPELTGSRQSFQKAMGNPCEFFVDIM

SEQ ID NO: 43 - protein, 736 aa, Homo sapiens
MAGSSTGGGGVGETKVIYHLDEEETPYLVKIPVPAERITLGDFKSVLQRPAGAKYFFKSMDQDFGVVKEEISDDNARLPCFNGRVVSWLVSSDNPQPEMAPPVHEPRAELAPPAPPLPPLPPERTSGIGDSRPPSFHPNVSSSHENLEPETETESVVSLRRERPRRRDSSEHGAGGHRTGGPSRLERHLAGYESSSTLMTSELESTSLGDSDEEDTMSRFSSSTEQSSASRLLKRHRRRRKQRPPRLERTSSFSSVTDSTMSLNIITVTLNMEKYNFLGISIVGQSNERGDGGIYIGSIMKGGAVAADGRIEPGDMLLQVNDMNFENMSNDDAVRVLRDIVHKPGPIVLTVAKCWDPSPQAYFTLPRNEPIQPIDPAAWVSHSAALTGTFPAYPGSSSMSTITSGSSLPDGCEGRGLSVHTDMASVTKAMAAPESGLEVRDRMWLKITIPNAFLGSDVVDWLYHHVEGFPERREARKYASGLLKAGLIRHTVNKITFSEQCYYVFGDLSGGCESYLVNLSLNDNDGSSGASDQDTLAPLPGATPWPLLPTFSYQYPAPHPYSPQPPPYHELSSYTYGGGSASSQHSEGSRSSGSTRSDGGAGRTGRPEERAPESKSGSGSESEPSSRGGSLRRGGEASGTSDGGPPPSRGSTGGAPNLRAHPGLHPYGPPPGMALPYNPMMVVMMPPPPPPVPPAVQPPGAPPVRDLGSVPPELTASRQSFHMAMGNPSEFFVDVM

SEQ ID NO: 44 - DNA, 108 bp, Unknown
  Antibody Light Chain Regions
gacattgtgctgacacagtctcctgcttccttagctgtatctctggggcagagggccaccatctcatacagggccagcaaaagtgtcagtacatctggctatagttat

SEQ ID NO: 45 - protein, 36 aa, Unknown
  Antibody Light Chain Regions
DIVLTQSPASLAVSLGQRATISYRASKSVSTSGYSY

SEQ ID NO: 46 - DNA, 60 bp, Unknown
  Antibody Light Chain Regions
atgcactggaaccaacagaaaccaggacagccacccagactcctcatctatcttgtatcc

SEQ ID NO: 47 - protein, 20 aa, Unknown
  Antibody Light Chain Regions
MHWNQQKPGQPPRLLIYLVS

SEQ ID NO: 48 - DNA, 21 bp, Unknown
  Antibody Light Chain Regions
aacctagaatctggggtccct

SEQ ID NO: 49 - protein, 7 aa, Unknown
  Antibody Light Chain Regions
NLESGVP

SEQ ID NO: 50 - DNA, 21 bp, Unknown
  Antibody Light Chain Regions
gccaggttcagtggcagtggg

SEQ ID NO: 51 - protein, 7 aa, Unknown
  Antibody Light Chain Regions
ARFSGSG

SEQ ID NO: 52 - DNA, 120 bp, Unknown
  Antibody Light Chain Regions
tctgggacagacttcaccctcaacatccatcctgtggaggaggaggatgctgcaacctattactgtcagcacattagggagcttacacgttcggaggggggaccaagctgaaaaaacggg

SEQ ID NO: 53 - protein, 39 aa, Unknown
  Antibody Light Chain Regions
SGTDFTLNIHPVEEEDAATYYCQHIRELTRSEGGPSKNG

SEQ ID NO: 54 - DNA, 108 bp, Unknown
  Antibody Light Chain Regions
gacattgtggtgacacagtctcctgcttccttagctgtatctctggggcagagggccaccatctcatacagggccagcaaaagtgtcagtacatctggctatagttat

SEQ ID NO: 55 - protein, 36 aa, Unknown
  Antibody Light Chain Regions
DIVVTQSPASLAVSLGQRATISYRASKSVSTSGYSY

SEQ ID NO: 56 - DNA, 123 bp, Unknown
  Antibody Light Chain Regions
tctgggacagacttcaccctcaacatccatcctgtggaggaggaggatgctgcaacctattactgtcagcacattagggagcttagcacgttncggaggggggagccaagctgaaataaacgg

SEQ ID NO: 57 - protein, 40 aa, Unknown
  Antibody Light Chain Regions
SGTDFTLNIHPVEEEDAATYYCQHIRELSTXRRGEPSNKR

SEQ ID NO: 58 - DNA, 21 bp, Unknown
  Antibody Light Chain Regions
aacctagaatctaagaagtca

SEQ ID NO: 59 - protein, 7 aa, Unknown
  Antibody Light Chain Regions
NLESKKS

SEQ ID NO: 60 - DNA, 21 bp, Unknown
  Antibody Light Chain Regions
cctgccaggttcagtggtcag

SEQ ID NO: 61 - protein, 7 aa, Unknown
  Antibody Light Chain Regions
PARFSGQ

SEQ ID NO: 62 - DNA, 134 bp, Unknown
  Antibody Light Chain Regions
tggtgtctggtgtacagacttcaccctcagacatccatgcctgtcggaggaggaggatgcctgcaacctgattatntgtgcagcacattagggagcttacacgttacggaggggggaccaagctgaaaaaacgg

SEQ ID NO: 63 - protein, 41 aa, Unknown
  Antibody Light Chain Regions
WCLVYRLHPQTSMPVGGGGCLQPDYXCSTLGSLHVTEGGPS

SEQ ID NO: 64 - DNA, 27 bp, Artificial Sequence
  Antibody Heavy Chain Regions
ngttncagcctgnaggagtcnggtgga

SEQ ID NO: 65 - DNA, 72 bp, Unknown
  Antibody Heavy Chain Regions
ggattggtgcagcctaaagggtcattgaaactctcatgtgcagcctctggattcacttttaatacctacgcc

SEQ ID NO: 66 - DNA, 102 bp, Unknown
  Antibody Heavy Chain Regions
atgaactgggtccgccaggctccaggaaagggtttggaatgggttgctcgcataagaactagacgttataattctgcaacatattatgccgattctgtgaaa

SEQ ID NO: 67 - DNA, 100 bp, Unknown
  Antibody Heavy Chain Regions
gacaggttcaccatctccagagatgattcacggggcatgctctatctgcaaatgaacaacttgaaaactgaggacacagccatgtattactgtgtgaggc

SEQ ID NO: 68 - DNA, 11 bp, Unknown
  Antibody Heavy Chain Regions
agtcnggacct

SEQ ID NO: 69 - DNA, 72 bp, Unknown
  Antibody Heavy Chain Regions
gagctggtgaagcctggggcttcagtgaagatgtcctgcaaggcttctggatacacattcactgactatgtt

SEQ ID NO: 70 - protein, 24 aa, Unknown
  Antibody Heavy Chain Regions
ELVKPGASVKMSCKASGYTFTDYV

SEQ ID NO: 71 - DNA, 75 bp, Unknown
  Antibody Heavy Chain Regions
ttaagctgggtgaagcagagaactggacagggccttgagtggattggagagatttatcctggatatggtagtact

SEQ ID NO: 72 - protein, 25 aa, Unknown
  Antibody Heavy Chain Regions
LSWVKQRTGQGLEWIGEIYPGYGST

SEQ ID NO: 73 - DNA, 21 bp, Unknown
  Antibody Heavy Chain Regions
tactacaatgagaagttcaag

SEQ ID NO: 74 - protein, 7 aa, Unknown
  Antibody Heavy Chain Regions
YYNEKFK

SEQ ID NO: 75 - DNA, 156 bp, Unknown
  Antibody Heavy Chain Regions
ggcaaggccacactgactgctgacaaatcctccaacacagcctacatgcagctcagcagcctgacatctgaggactctgcggtctatttctgtgcaagatggggggattgcttttgcttatctggggccaaggganctctggtcanctgtctctgc

SEQ ID NO: 76 - protein, 52 aa, Unknown
  Antibody Heavy Chain Regions
GKATLTADKSSNTAYMQLSSLTSEDSAVYFCARWGDCFCLSGAKGXLVXCLC

SEQ ID NO: 77 - protein, 12 aa, Homo sapiens
HNNEAGRTTVFS

SEQ ID NO: 78 - protein, 14 aa, Homo sapiens
NVASSTNLLTDSKS

SEQ ID NO: 79 - protein, 5 aa, Artificial Sequence
  Synthetic peptide linker
GGGGS

SEQ ID NO: 80 - protein, 12 aa, Artificial Sequence
  Synthetic polypeptide
HNNEAGRTTVFS

(19) United States
(12) Patent Application Publication
(10) Pub. No.: US 2009/0304695 A1
(43) Pub. Date: Dec. 10, 2009

(54) METHODS FOR TREATING CANCER BY INHIBITING WNT SIGNALING

(75) Inventors: Biao He, South San Francisco, CA (US); Liang You, San Francisco, CA (US); Zhidong Xu, San Francisco, CA (US); David M. Jablons, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: REGENTS OF THE UNIVERSITY OF CALIFORNIA, Oakland, CA (US)

(21) Appl. No.: 12/363,569

(22) Filed: Jan. 30, 2009

Related U.S. Application Data
(62) Division of application No. 10/678,639, filed on Oct. 3, 2003, now abandoned.
(60) Provisional application No. 60/509,037, filed on Oct. 4, 2002.
(60) Provisional application No. 60/491,350, filed on Jul. 31, 2003.

(51) Int. Cl. A61K 39/395; A61P 35/00

(52) U.S. Cl. 424/135.1; 424/172.1; 424/133.1

(57) ABSTRACT

This invention relates to methods of inhibiting the growth of cancer cells that overexpress a Wnt protein. The methods comprise contacting the cell with an agent that inhibits binding of the Wnt protein to a Frizzled receptor.

### CROSS-REFERENCES TO RELATED APPLICATIONS

This application claims benefit of U.S. provisional application No. 60/491,350 filed Jul. 31, 2003 and claims benefit of U.S. provisional application No. ______ filed Oct. 4, 2002 (converted from non-provisional application No. 10/264,825). Each application is incorporated by reference herein.

### FIELD OF THE INVENTION

This invention relates to methods of inhibiting the growth of cancer cells that overexpress a Wnt protein. The methods comprise contacting the cell with an agent that inhibits binding of the Wnt protein to a Frizzled receptor.

### BACKGROUND OF THE INVENTION

The Wingless-type (Wnt) Frizzled protein receptor pathway involves important regulatory genes that carry polymorphisms associated with primary carcinomas. In the course of downstream signaling cytosolic β-catenin accumulates, translocates into the nucleus, and then enhances gene expression by complexing with other transcription factors Uthoff et al., Mol Carcinog, 31:56-62 (2001). In the absence of Wnt signals, free cytosolic β-catenin is incorporated into a complex consisting of Axin, the adenomatous polyposis coli (APC) gene product, and glycogen synthase kinase (GSK)-3β. Conjunctional phosphorylation of Axin, APC, and β-catenin by GSK-3β designates β-catenin for the ubiquitin pathway and degradation by proteasomes Uthoff et al., Mol Carcinog, 31:56-62 (2001); Matsuzawa et al., Mol Cell, 7:915-926 (2001).

Disheveled (Dvl) is a positive mediator of Wnt signalling positioned downstream of the frizzled receptors and upstream of βcatenin. GSK-3 phosphorylates several proteins in the Wnt pathway and is instrumental in the downstream regulation of βcatenin. Mutations in the gene APC are an initiating event for both sporadic and hereditary colorectal tumorigenesis. APC mutants are relevant in tumorigenesis, since the aberrant protein is an integral part of the Wnt-signaling cascade. The protein product contains several functional domains acting as binding and degradation sites for catenin. Mutations that occur in the amino-terminal segment of βcatenin are usually involved in phosphorylation-dependent, ubiquitin-mediated degradation and, thus, stabilize βcatenin. When stabilized cytoplasmic-catenin accumulates, it translocates to the nucleus interacting with the Tcf/Lef high-mobility group of transcription factors that modulate expression of oncogenes such as c-myc.

It is known that Wnt/β-catenin signaling promotes cell survival in various cell types Orford et al., J Cell Biol, 146:855-868 (1999); Cox et al., Genetics, 155:1725-1740 (2000); Reya et al., Immunity, 13:15-24 (2000); Satoh et al., Nat Genet, 24:245-250 (2000); Shin et al., Journal of Biological Chemistry, 274:2780-2785 (1999); Chen et al., J Cell Biol, 152:87-96 (2001); Ioannidis et al., Nat Immunol, 2:691-697 (2001). Wnt signaling pathway is also thought to be associated with tumor development and/or progression (Polakis et al., Genes Dev, 14:1837-1851 (2000); Cox et al., Genetics, 155:1725-1740 (2000); Bienz et al., Cell, 103:311-320 (2000); You et al., J Cell Biol, 157:429-440 (2002)). Aberrant activation of the Wnt signaling pathway is associated with a variety of human cancers, correlating with the over-expression or amplification of c-Myc (Polakis et al., Genes Dev, 14:1837-1851 (2000); Bienz et al., Cell, 103:311-320 (2000); Brown et al., Breast Cancer Res, 3:351-355 (2001); He et al., Science, 281:1509-1512 (1998); Miller et al., Oncogene, 18:7860-7872 (1999). In addition, c-Myc was identified as one of the transcriptional targets of the β-catenin/Tcf in colorectal cancer cells (He et al., Science, 281:1509-1512 (1998); de La Coste et al., Proc Natl Acad Sci USA, 95:8847-8851 (1998); Miller et al., Oncogene, 18:7860-7872 (1999); You et al., J Cell Biol, 157:429-440 (2002)).

In addition to the Wnt ligands, a family of secreted Frizzled-related proteins (sFRPs) has been isolated. sFRPs appear to function as soluble endogenous modulators of Wnt signaling by competing with the membrane-spanning Frizzled receptors for the binding of secreted Wnt ligands (Melkonyan et al., Proc Natl Acad Sci USA, 94:13636-13641 (1997)). sFRPs can either antagonize Wnt function by binding the protein and blocking access to its cell surface signaling receptor, or they can enhance Wnt activity by facilitating the presentation of ligand to the Frizzled receptors Uthoff et al., Mol Carcinog, 31:56-62 (2001). Another protein called Dickkopf (Dkk) is also found to interfere with Wnt signaling and diminish accumulation of cytosolic β-catenin (Fedi et al., J Biol Chem, 274:19465-19472 (1999); Moon et al., Cell, 88:725-728 (1997)). Dkk-1 antagonizes Wnt-induced signals by binding to a LDL-receptor-related protein 6 (LRP6) adjacent to the Frizzled receptor (Nusse et al., Nature, 411:255-256 (2001)). Recently H. Suzuki, et al. found that sFRPs are hypermethylated with a high frequency in colorectal cancer cell lines and this hypermethylation is associated with a lack of basal sFRP expression (Suzuki et al., Nat Genet, 31:141-149 (2002)). Over-expression of Dkk-1 is also found to sensitize brain tumor cells to apoptosis (Shou et al., Oncogene, 21:878-889 (2002)).

Despite recent advances in the understanding of Wnt signaling, the role of this pathway in oncogenesis is unclear. Thus, the prior art fails to provide clear evidence that compounds that modulate this pathway could be useful for treatment of cancer. The present invention addresses these and other needs.

### BRIEF SUMMARY OF THE INVENTION

This invention provides methods of inhibiting the growth of a cancer cell that overexpresses a Wnt protein. The methods comprising contacting the cell with an agent that inhibits binding of the Wnt protein to a Frizzled receptor.

In some embodiments, the agent is an antibody. For example, the antibody can specifically binds to a Wnt protein, Wnt-1 or Wnt-2. In other embodiments the antibody specifically binds a Frizzled receptor, such as Frizzled1, Frizzled2, Frizzled3, Frizzled4, Frizzled5, Frizzled6, Frizzled7, Frizzled8, Frizzled9 and Frizzled10 receptor.

Antibodies of the invention can be monoclonal antibodies and can be prepared and modified in a number of ways. For example, the antibody may be recombinantly produced. In some embodiments, the antibody is a humanized antibody or a single chain Fv fragment (scFv).

The invention also provides therapeutic methods of treating cancer. In these embodiments, the cancer cell is in a patient and the step of contacting is carried out by administering the agent to the patient. The method may further comprise administering to the patient a second therapeutic agent, such as a chemotherapeutic agent or radiation therapy. The cancer cell may be a breast cancer cell, colorectal cancer cell, a lung cancer cell, a sarcoma cell, or a mesothelioma cell, a prostate cancer cell, a pancreatic cancer cell, a cervical cancer cell, an ovary cancer cell, a gastric cancer cell, an esophageal cancer cell, a head and neck cancer cell, a hepatocellular carcinoma cell, a melanoma cell, a glioma cell, or a glioblastoma cell.

The invention also provides pharmaceutical compositions comprising a pharmaceutically acceptable excipient and a monoclonal antibody that specifically binds Wnt a Wnt or Frizzled protein, for example a Wnt1 protein. The antibody can be further conjugated to an effector component, such as label, a radioisotope or a cytotoxic chemical.

In another aspect, the invention provides a method of screening for an agent that inhibits the proliferation of a cancer cell, the method comprising contacting the agent with a Dvl protein or nucleic acid, determining Dvl protein activity or expression, and identifying a compound that inhibits Dvl protein acitivty or expression, thereby identifying an agent that inhibits the proliferation of a cancer cell. The method can further comprise contacting an identified compound with a cancer cell, and selecting the compound that inhibits proliferation of the cancer cell. In some embodiments, the cancer cell is a lung cancer cell or a mesothelioma cell.

The invention also provides a method of inhibiting the growth of a cancer cell that overexpresses a Dvl protein, the method comprising contacting the cell with an agent that inhibits Dvl expression or activity. In some embodiments, the cancer cell is a lung cancer cell or a mesothelioma cell. The agent can be, e.g., a small molecule or an siRNA.

### Definitions

The terms “Wnt protein” or “Wnt ligand” refer to a family of mammalian proteins related to the Drosophila segment polarity gene, wingless. In humans, the Wnt family of genes typically encode 38 to 43 kDa cysteine rich glycoproteins having hydrophobic signal sequence, and a conserved asparagine-linked oligosaccharide consensus sequence (see e.g., Shimizu et al Cell Growth Differ 8:1349-1358 (1997)). The Wnt family contains at least 16 mammalian members. Exemplary Wnt proteins include Wnt-1, Wnt-2, Wnt-3, Wnt-3A, Wnt-4, Wnt-5A, Wnt-6, Wnt-7A, Wnt-7B, Wnt-8A, Wnt-8B, Wnt-10B, Wnt-11, Wnt-13, Wnt 14, Wnt 15, and Wnt 16. The sequence of some exemplary Wnt proteins of the invention are set forth in the sequence listing. In addition, overexpression of particular Wnt proteins have been shown to be associated with certain cancers. For example, WNT-2 is overexpressed in gastric and colorectal cancer, (Katoh et al., Int J Oncol, 19:1003-1007 (2001)); Wnt-1 is overexpressed in head and neck cancer, and WNT-5A and Wnt-8B are overexpressed in gastric cancer, (Saitoh et al., Int J Mol Med, 9:515-519 (2002); Saitoh et al., Int J Oncol, 20:343-348 (2002)).

The terms “frizzled protein” or “frizzled receptor” refer to a family of mammalian proteins related to the Drosophila frizzled genes, which play a role in the development of tissue polarity. The Frizzled family comprises at least 10 mammalian genes. Exemplary human Frizzled receptors include Frizzled1, Frizzled2, Frizzled3, Frizzled4, Frizzled5, Frizzled6, Frizzled7, Frizzled8, Frizzled9 and Frizzled10. The sequence of exemplary Frizzled receptors are set forth in the sequence listing. The mammalian homologues of the Drosophila frizzled protein share a number of common structural motifs. The N terminus located at the extracellular membrane surface is followed by a signal sequence, a domain of 120 amino acids with an invariant pattern of 10 cysteine residues, and a highly divergent region of 40-100 largely variable hydrophilic amino acids. Putative hydrophobic segments form seven membrane-spanning helices linked by hydrophilic loops, ending with the C terminus located at the intracellular face of the membrane. The cysteine-rich domains (CRDs) and the transmembrane segments are strongly conserved, suggesting a working model in which an extracellular CRD is tethered by a variable linker region to a bundle of seven membrane-spanning -helices. Frizzled protein receptors are, therefore, involved in a dynamic model of transmembrane signal transduction analogous to G-protein-coupled receptors with amino-terminal ligand binding domains. Frizzled1, Frizzled2, and Frizzled7 in lung and colorectal cancers, Sagara et al., Commun, 252:117-122 (1998); Frizzled3 in human cancer cells including lung, cervical and colorectal cancers, (Kirikoshi et al., Biochem Biophys Res Commun, 271:8-14 (2000)); Frizzled7 in gastric cancer (Kirikoshi et al., Int J Oncol, 19:111-115 (2001)); Frizzled10 in gastric and colorectal cancer (Kirikoshi et al., Int J Oncol, 19:767-771 (2001); Terasaki et al., Int J Mol Med, 9:107-112 (2002)).

In addition to the Wnt ligands, a family of secreted frizzled-related proteins (sFRPs) has been isolated. sFRPs appear to function as soluble endogenous modulators of Wnt signaling by competing with the membrane-spanning frizzled receptors for the binding of secreted Wnt ligands. sFRPs, therefore, modulate apoptosis susceptibility, exerting an antagonistic effect on programmed cell death. sFRPs can either antagonize Wnt function by binding the protein and blocking access to its cell surface signaling receptor, or they can enhance Wnt activity by facilitating the presentation of ligand to the frizzled receptors. To date, sFRPs have not yet been linked causatively to cancer.

The term “Dishevelled” or “Dvl” refer to a member of a family of Dishevelled proteins, the full-length sequences of which typically possess three conserved domains, a DIX domain, present in the Wnt antagonizing protein Axin; a PDZ domain involved in protein-protein interactions, and a DEP domain found in proteins that regulate Rho GTPases. Dvl proteins include, for example, Dvl-1, Dvl-2, and Dvl-3. Nucleic acid and protein Dvl sequence are known from a variety of species, including mouse and human. Exemplary human Dvl-1, Dvl-2, and Dvl-3 protein sequences are available under reference sequences NP_{—}004412, NP_{—}004413, and NM_{—}004414, respectively.

“Inhibitors” of Wnt signaling refers to compounds that, e.g., bind to Wnt or Frizzled proteins, or partially or totally block Wnt signaling as measured in known assays for Wnt signaling (e.g., measurement of β catenin levels, or oncogene expression controlled by Tcf and Lef transcription factors). Inhibitors, include modified versions of Wnt or Frizzled proteins, as well as naturally occurring and synthetic ligands, antagonists, agonists, antibodies, small chemical molecules, and the like. Assays for detecting inhibitors of the invention are described in more detail below.

A “cancer cell that overexpresses a Wnt protein” is a cancer cell in which expression of a particular Wnt protein is at least about 2 times, usually at least about 5 times the level of expression in a normal cell from the same tissue. Methods for determining the level of expression of a particular gene are well known in the art. Such methods include RT-PCR, use of antibodies against the gene products, and the like.

As used herein, “antibody” includes reference to an immunoglobulin molecule immunologically reactive with a particular antigen, and includes both polyclonal and monoclonal antibodies. The tern also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or a “VH” refer to the variable region of an immunoglobulin heavy chain of an antibody, including the heavy chain of an Fv, scFv, or Fab. References to “V_{L}” or a “VL” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

“Biological sample” as used herein is a sample of biological tissue or fluid that contains nucleic acids or polypeptides, e.g., of a Wnt protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of (-sheet and (-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The radioisotope may be, for example, 3H, 14C, 32P, 35S, or 125I. In some cases, particularly using antibodies against the proteins of the invention, the radioisotopes are used as toxic moieties, as described below. The labels may be incorporated into the nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). The lifetime of radiolabeled peptides or radiolabeled antibody compositions may extended by the addition of substances that stablize the radiolabeled peptide or antibody and protect it from degradation. Any substance or combination of substances that stablize the radiolabeled peptide or antibody may be used including those substances disclosed in U.S. Pat. No. 5,961,955.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background.

Specific binding to an antibody under such conditions requires an antibody that is selected for its specificity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with Wnt or Frizzled proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

“Tumor cell” refers to precancerous, cancerous, and normal cells in a tumor.

“Cancer cells,” “transformed” cells or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. In the present invention transformation is typically associated with overexpression of Wnt and/or Frizzled proteins. Transformation is associated with other phenotypic changes, such as immortalization of cells, aberrant growth control, nonmorphological changes, and/or malignancy (see, Freshney, Culture of Animal Cells a Manual of Basic Technique (3rd ed. 1994)).

### BRIEF DESCRIPTION OF THE DRAWINGS

FIG. 1 shows that anti-Wnt-1 or anti-Wnt-2 antibody specifically induces apoptosis in various human cancer cell lines.

FIG. 2 shows the fraction of apoptotic cell death (%) after anti-Wnt antibody treatment.

FIG. 3A shows that anti-Wnt antibody-induced apoptosis is correlated with the Wnt expression in various cancer cell lines. FIG. 3B shows the effect of Wnt blocking peptides on anti-Wnt antibody-induced apoptosis.

FIG. 4 shows a time course (FIG. 4A) and dosage cures of anti-Wnt antibody-induced apoptosis in lung cancer cell lines (FIG. 4B).

FIG. 5 shows that anti-Wnt-1 monoclonal antibody induces apoptosis in different human cancer cell lines in vitro. a. 0.5% Crystal Violet staining of cancer cells MCF-7 (upper two rows) about 48 hrs and H460 (bottom two rows) about 72 hrs after control or the anti-Wnt-1 monoclonal antibody treatment. Concentrations of the control or anti-Wnt-1 antibodies used from left to right are 0.0, 1.0 and 10.0 μg/ml, respectively. b. Example of apoptosis analysis by flow cytometry. From top to bottom, H460 cancer cells were treated with 5.0 μg/ml of control antibody, 1.0 μg/ml and 5.0 μg/ml of anti-Wnt-1 antibody, respectively, for about 72 hrs. FL1-H represents Annexin V-FITC staining and FL3-H represents propidium iodide (PI) staining. c. Dose responses of H460 and MCF-7 cancer cells to monoclonal antibody treatment. Measurements were taken after 72 hrs of incubation for H460 and 48 hrs of incubation for MCF-7. Squares (□) and circles (∘) represent fraction of cell death in MCF-7 and H460 cells treated with anti-Wnt-1 antibody, respectively. Diamonds (⋄) and triangles (Δ) represent fraction of cell death in MCF-7 and H460 cells treated with control antibody, respectively. Results are the means ±SD (error bars).

FIGS. 6A-6C show that an anti-Wnt-1 monoclonal antibody suppresses tumor growth in vivo.

FIG. 7 shows the sequences of heavy and light chain regions of monoclonal antibodies generated to peptides equences set forth in SEQ ID NO:2, SEQ ID NO:4, or SEQ ID NO:9.

FIG. 8 shows that ΔPDZ-Dvl inhibited the tumorigenesis of mesothelioma cell in vivo. ΔPDZ-Dvl-transfected malignant pleural mesothelioma LRK1A and REN cells were unable to grow after subcutaneous (s.c.) injection in athymic mice compared with empty vector-trasnfected controls. Results are the means ±SD (bars) for five animals in each group.

FIG. 9 shows suppression of NCI-H1703 growth by the Dvl siRNA. Cells (3×10⁴) were plated in 24-2311 plates and transfected with the Dvl siRNA (sequares) or the contro si RNA (circles). After 72 h of transfection, viable cells (trypan blue exclusion were collected every 24 h by trypsinization and counted. Notably, after 72 h of transfection, cell growth was significantly suppressed (P<0.05).

FIG. 10 shows that over-expression of Wnt signal antagonist FRP or DKK induces apoptosis in cancer cells.

### DETAILED DESCRIPTION

This invention is based on the discovery that Wnt-Fz signaling pathway plays a role in oncogenesis. It is known that Wnt proteins often have high level expression in cancer. However, little is known regarding Wnt-Fz signaling modification of the cell death machinery in cancer. The present disclosure provides evidence that inhibitors of Wnt signaling can induce significant apoptosis in a number of cancer cells. The invention is useful for any cancer in which Wnt-Fz signaling affects cancer cell growth or survival. The invention is useful for treating cancers such as breast cancer, colorectal cancer, lung cancer, sarcoma, mesothelioma, prostate cancer, pancreatic cancer,cervical cancer, ovarian cancer, gastric cancer, esophageal cancer, head and neck cancer, hepatocellular carcinoma, melanoma, glioma, or glioblastoma.

Blocking Wnt signaling is shown here to lead to down-regulation of downstream components of the Wnt-Fz pathway, in particular, Dishevelled (Dvl) and β-catenin. Evidence provided here also shows that antibody-induced apoptosis occurs through activation of JNK, releasing Smac/Diablo and cytochrome C from mitochondria to the cytosol. Cytochrome C inactivates survinin, an inhibitor of apoptosis, that leads to the activation of caspases. The disclosure further provides evidence that monoclonal anti-Wnt-1 antibodies can suppress growth of tumors in vivo.

### Antibodies to WNT and Frizzled Proteins

As noted above, the invention provides methods of inhibiting Wnt signaling in cancer cells. In some embodiments of the invention, antibodies are used to block the binding between Wnt ligand and the Frizzled receptor. The antibodies can be raised against either Wnt or Frizzled proteins.

Methods of preparing polyclonal antibodies are known to the skilled artisan (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-16 fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In some embodiments, a monoclonal antibody is used. A preferred embodiment is a monoclonal antibody that binds the same epitope as the monoclonal antibody described in Example 11. The ability of a particular antibody to recognize the same epitope as another antibody is typically determined by the ability of one antibody to competitively inhibit binding of the second antibody to the antigen. Any of a number of competitive binding assays can be used to measure competition between two antibodies to the same antigen. For example, a sandwich ELISA assay can be used for this purpose. This is carried out by using a capture antibody to coat the surface of a well. A subsaturating concentration of tagged-antigen is then added to the capture surface. This protein will be bound to the antibody through a specific antibody:epitope interaction. After washing a second antibody, which has been covalently linked to a detectable moeity (e.g., HRP, with the labeled antibody being defined as the detection antibody) is added to the ELISA. If this antibody recognizes the same epitope as the capture antibody it will be unable to bind to the target protein as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

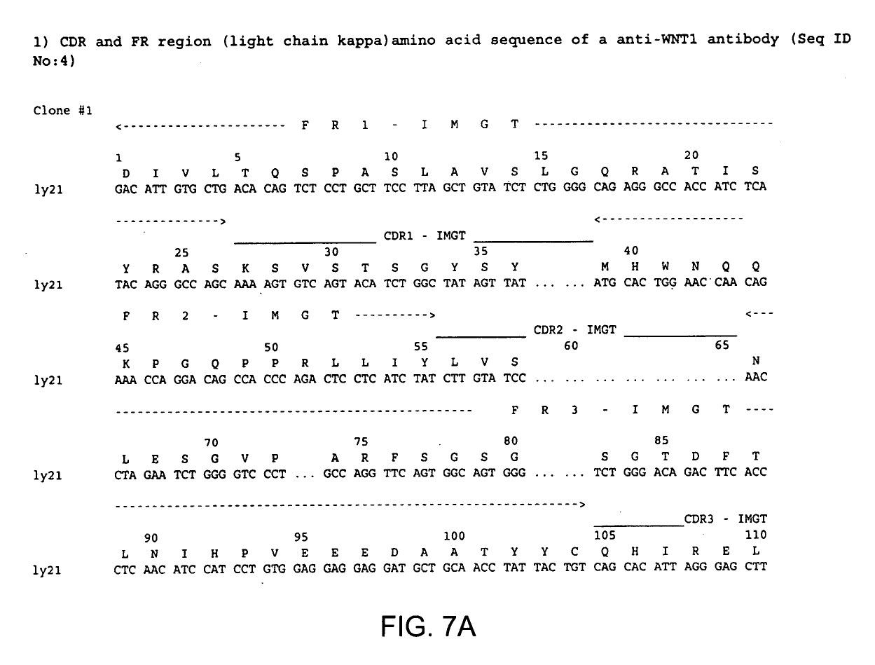

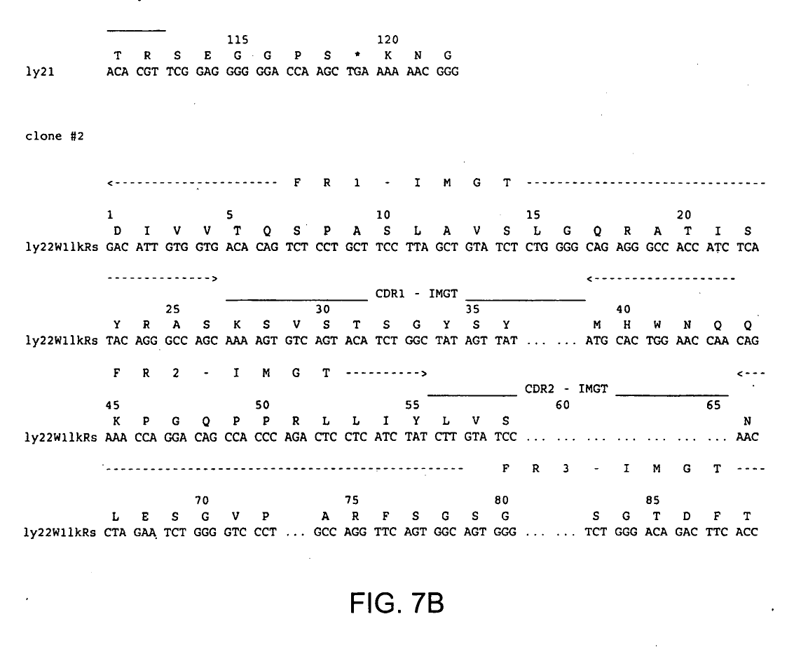

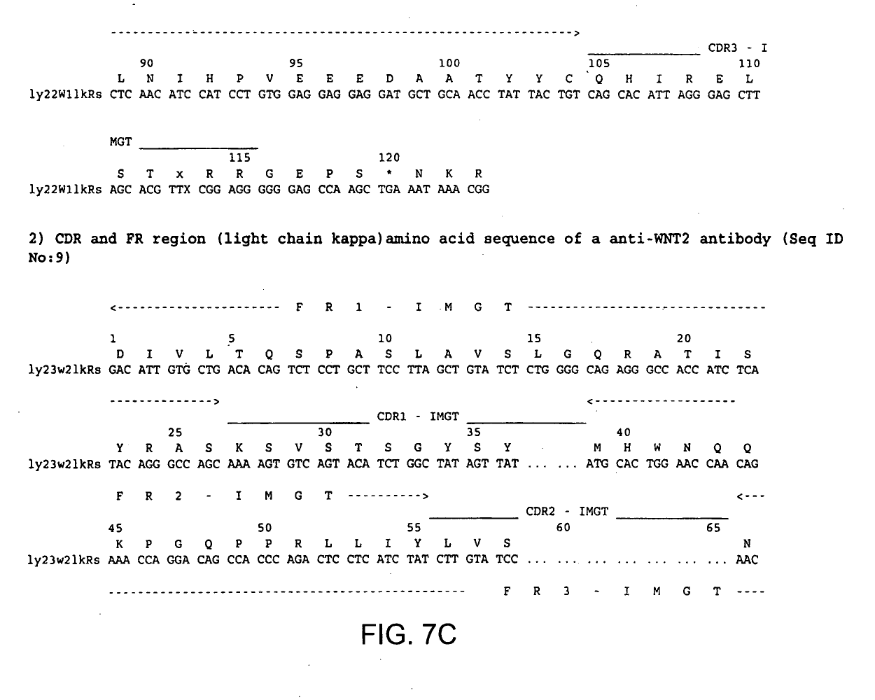

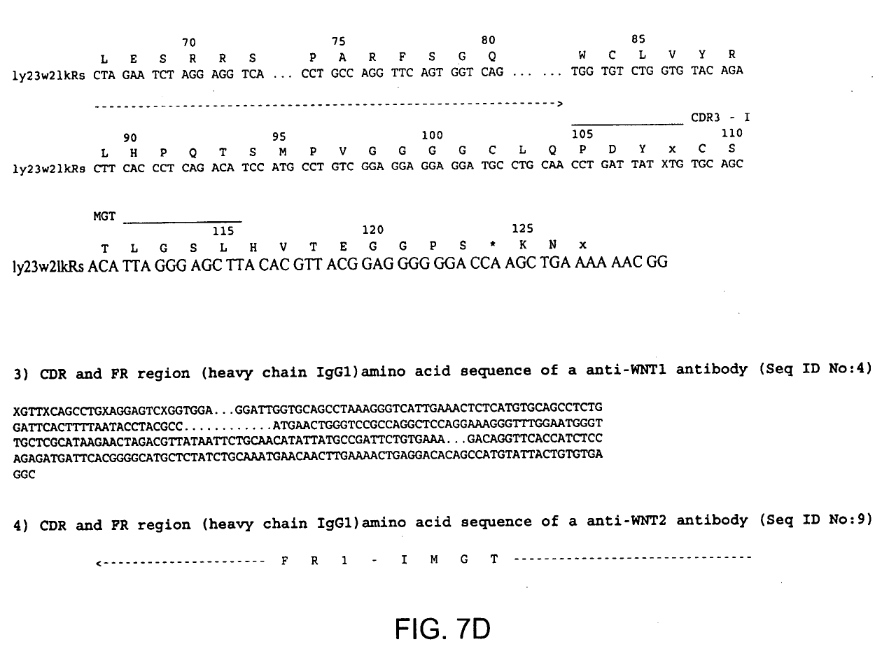

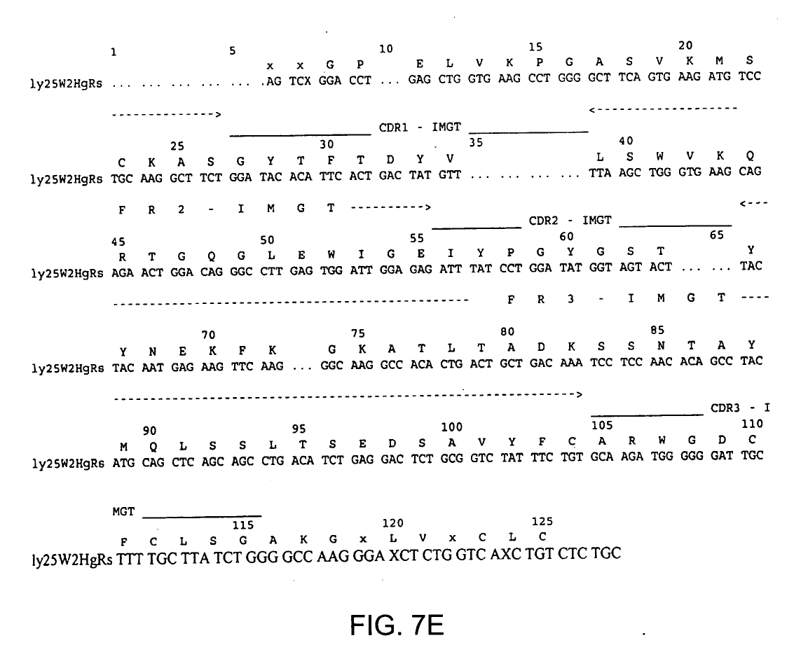

In some embodiments, a monoclonal anti-Wnt antibody of the invention binds to amino acids 201-212 of human Wnt-l (HNNEAGRTTVFS), amino acids 39-52 of human Wnt-1 (NVASSTNLLTDSKS), or amino acids 49-63 of human Wnt-2 (SSQRQLCHRHPDVMR). For example, such a monoclonal antibody may have the binding specificity (i.e., in this context, the same CDRs, or substantially the same CDRs) of an antibody having V_{H} and V_{L} chains as set forth in FIG. 7. An antibody of the invention may therefore comprises a CDR as set forth in a V_{H} or V_{L} sequence shown in FIG. 7 and, additionally, may have at least 80% identity, preferably, 85%, 90%, or 95% identity to the V_{H} or V_{L} sequence. For example, in particular embodiments, the antibody may comprise the CDRs of a V_{H} and V_{L} sequence of FIG. 7 and human framework sequences.

In some embodiments the antibodies to the Wnt or Frizzled proteins are chimeric or humanized antibodies. As noted above, humanized forms of antibodies are chimeric immunoglobulins in which residues from a complementary determining region (CDR) of human antibody are replaced by residues from a CDR of a non-human species such as mouse, rat or rabbit having the desired specificity, affinity and capacity.

Human antibodies can be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

In some embodiments, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993). Generally the peptide linker will have no specific biological activity other than to join the regions or to preserve some minimum distance or other spatial relationship between the V_{H} and V_{L}. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Gly-Ser, preferably 2, 3, 4, 5, or 6 such sequences. However, it is to be appreciated that some amino acid substitutions within the linker can be made. For example, a valine can be substituted for a glycine.

Methods of making scFv antibodies have been described. See, Huse et al., supra; Ward et al. supra; and Vaughan et al., supra. In brief, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell. The scFv that specifically bind to the desired antigen are typically found by panning of a phage display library. Panning can be performed by any of several methods. Panning can conveniently be performed using cells expressing the desired antigen on their surface or using a solid surface coated with the desired antigen. Conveniently, the surface can be a magnetic bead. The unbound phage are washed off the solid surface and the bound phage are eluted.

Regardless of the method of panning chosen, the physical link between genotype and phenotype provided by phage display makes it possible to test every member of a cDNA library for binding to antigen, even with large libraries of clones.

In some embodiments, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for the Wnt or Frizzled protein, the other one is for another cancer antigen. Alternatively, tetramer-type technology may create multivalent reagents.

In some embodiments, the antibody is conjugated to an effector moiety. The effector moiety can be any number of molecules, including labeling moieties such as radioactive labels or fluorescent labels, or can be a therapeutic moiety. If the effector moiety is a therapeutic moiety, it will typically be a cytotoxic agent. In this method, targeting the cytotoxic agent to cancer cells, results in direct killing of the target cell. This embodiment is typically carried out using antibodies against the Frizzled receptor. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, auristatin and the like. Cytotoxic agents also include radiochemicals made by conjugating radioisotopes to antibodies raised against Wnt or Frizzled proteins, or binding of a radionuclide to a chelating agent that has been covalently attached to the antibody.

### Binding Affinity of Antibodies of the Invention

Binding affinity for a target antigen is typically measured or determined by standard antibody-antigen assays, such as Biacore competitive assays, saturation assays, or immunoassays such as ELISA or RIA.

Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is <1 μM, preferably <100 nM, and most preferably <0.1 nM. Antibody molecules will typically have a K_{D} in the lower ranges. K_{D}=[Ab−Ag]/(Aberle et al., EMBO Journal, 16:3797-3804 (1997)) where (Aberle et al., EMBO Journal, 16:3797-3804 (1997)) is the concentration at equilibrium of the antibody, (Aberle et al., EMBO Journal, 16:3797-3804 (1997)) is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

The antibodies of the invention specifically bind to Wnt or Frizzled proteins. By “specifically bind” herein is meant that the antibodies bind to the protein with a K_{D} of at least about 0.1 mM, more usually at least about 1 μM, preferably at least about 0.1 μM or better, and most preferably, 0.01 μM or better.

### Diagnostic Assays

The present invention also provides diagnostic assays for detecting Wnt or Frizzled over-expression. As noted above over-expression of these genes can be used to identify cancer cells. In preferred embodiments, activity of the Wnt or Frizzled gene of interest is determined by a measure of gene transcript (e.g. mRNA), by a measure of the quantity of translated protein, or by a measure of gene product activity.

Methods of detecting and/or quantifying the gene transcript (mRNA or cDNA) using nucleic acid hybridization techniques are known to those of skill in the art. For example, one method for evaluating the presence, absence, or quantity of mRNA involves a Northern blot transfer.

The probes can be full length or less than the full length of the nucleic acid sequence encoding the protein. Shorter probes are empirically tested for specificity. Preferably nucleic acid probes are 20 bases or longer in length. Visualization of the hybridized portions allows the qualitative determination of the presence or absence of mRNA.

In another preferred embodiment, a transcript (e.g., mRNA) can be measured using amplification (e.g. PCR) based methods as described above for directly assessing copy number of DNA. In a preferred embodiment, transcript level is assessed by using reverse transcription PCR (RT-PCR).

The “activity” of a Wnt or Frizzled gene can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like. The isolated proteins can also be sequence according to standard techniques to identify polymorphisms.

The antibodies of the invention can also be used to detect Wnt or Frizzled proteins, or cells expressing them, using any of a number of well recognized immunological binding assays (see, e.g., U.S. Pat. Nos. 4,366,241; 4,376,110; 4,517,288; and 4,837,168). For a review of the general immunoassays, see also Methods in Cell Biology, Vol. 37, Asai, ed. Academic Press, Inc. New York (1993); Basic and Clinical Immunology 7th Edition, Stites & Terr, eds. (1991).

Thus, the present invention provides methods of detecting cells that over-express Wnt or Frizzled proteins. In one method, a biopsy is performed on the subject and the collected tissue is tested in vitro. The tissue or cells from the tissue is then contacted, with an anti-Wnt or anti-Frizzled antibody of the invention. Any immune complexes which result indicate the presence of the target protein in the biopsied sample. To facilitate such detection, the antibody can be radiolabeled or coupled to an effector molecule which is a detectable label, such as a radiolabel. In another method, the cells can be detected in vivo using typical imaging systems. Then, the localization of the label is determined by any of the known methods for detecting the label. A conventional method for visualizing diagnostic imaging can be used. For example, paramagnetic isotopes can be used for MRI. Internalization of the antibody may be important to extend the life within the organism beyond that provided by extracellular binding, which will be susceptible to clearance by the extracellular enzymatic environment coupled with circulatory clearance.

### Identification of Inhibitors of WNT Signaling

Wnt or Frizzled proteins (or cells expressing them) or members of the Wnt signaling pathway, e.g., dvl, can also be used in drug screening assays to identify agents that inhibit Wnt signaling. The present invention thus provides novel methods for screening for compositions which inhibit cancer.

Assays for Wnt signaling can be designed to detect and/or quantify any part of the Wnt signaling pathway. For example the ability of an agent to affect intracellular β-catenin levels or to induce aoptosis in target cells can be measured. Assays suitable for these purposes are described below.

Assays may include those designed to test binding activity to either the Wnt ligand, the Frizzled receptor, or another member of the Wnt signaling cascade, e.g., dvl. These assays are particularly useful in identifying agents that modulate Wnt activity. Virtually any agent can be tested in such an assay. Such agents include, but are not limited to natural or synthetic polypeptides, antibodies, natural or synthetic small organic molecules, nucleic acids and the like.

As noted above, a family of secreted Frizzled-related proteins (sFRPs) function as soluble endogenous modulators of Wnt signaling by competing with Frizzled receptors for the binding of secreted Wnt ligands. Thus, in some format, test agents are based on natural ligands (e.g., Wnts ligands or sFRPs) of the Frizzled receptor.

Any of the assays for detecting Wnt signaling are amenable to high throughput screening. High throughput assays binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

Other assays useful in the present invention are those designed to test neoplastic phenotypes of cancer cells. These assays include cell growth on soft agar; anchorage dependence; contact inhibition and density limitation of growth; cellular proliferation; cell death (apoptosis); cellular transformation; growth factor or serum dependence; tumor specific marker levels; invasiveness into Matrigel; tumor growth and metastasis in vivo; mRNA and protein expression in cells undergoing metastasis, and other characteristics of cancer cells.

The ability of test agents to inhibit cell growth can also be assessed by introducing the test into an animal model of disease, and assessing the growth of cancer cells in vivo. For example, human tumor cells can be introduced into an immunocompromised animal such as a “nude mouse”. The test agent (e.g., a small molecule or an antibody) is administered to the animal and the ability of the tumor cell to form tumors—as assessed by the number and/or size of tumors formed in the animal—is compared to tumor growth in a control animal without the agent.

### Inhibitors of Gene Expression

In one aspect of the present invention, inhibitors of the Wnt signaling pathway, e.g., Dvl inhibitors, can comprise nucleic acid molecules that inhibit expression of the target protein in the pathway. Conventional viral and non-viral based gene transfer methods can be used to introduce nucleic acids encoding engineered polypeptides, e.g., dominant negative forms of the protein, in mammalian cells or target tissues, or alternatively, nucleic acids e.g., inhibitors of target protein expression, such as siRNAs or anti-sense RNAs. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217(1993); Mitani & Caskey, TIBTECH 11:162-166(1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1: 13-26 (1994).

In some embodiments, small interfering RNAs are administered. In mammalian cells, introduction of long dsRNA (>30 nt) often initiates a potent antiviral response, exemplified by nonspecific inhibition of protein synthesis and RNA degradation. The phenomenon of RNA interference is described and discussed, e.g., in Bass, Nature 411:428-29 (2001); Elbahir et al., Nature 411:494-98 (2001); and Fire et al., Nature 391:806-11 (1998), where methods of making interfering RNA also are discussed. The siRNA inhibitors are less than 100 base pairs, typically 30 bps or shorter, and are made by approaches known in the art. Exemplary siRNAs according to the invention can have up to 29 bps, 25 bps, 22 bps, 21 bps, 20 bps, 15 bps, 10 bps, 5 bps or any integer thereabout or therebetween.

### Non-Viral Delivery Methods

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Viral Delivery Methods

The use of RNA or DNA viral based systems for the delivery of inhibitors of target Wnt pathway proteins, e.g., Dvl, are known in the art. Conventional viral based systems for the delivery of such nucleic acid inhibitors can include retroviral, lentivirus, adenoviral, adeno-associated and herpes simplex virus vectors for gene transfer.

In many gene therapy applications, it is desirable that the gene therapy vector be delivered with a high degree of specificity to a particular tissue type, e.g., a lung cancer. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient.

Ex vivo cell transfection for diagnostics, research, or for gene therapy (e.g., via re-infusion of the transfected cells into the host organism) is well known to those of skill in the art. In some embodiments, cells are isolated from the subject organism, transfected with inhibitor nucleic acids and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Vectors (e.g., retroviruses, adenoviruses, liposomes, etc.) containing therapeutic nucleic acids can also be administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington's Pharmaceutical Sciences, 17th ed., 1989).

### Kits use in Diagnostic, Research, and Therapeutic Applications

As noted above, the invention provides evidence of the overexpression of particular Wnt or Frizzled proteins in certain cancers. Thus, kits can be used for the detection of the particular nucleic acids or proteins disclosed here. In diagnostic and research applications such kits may include any or all of the following: assay reagents, buffers, Wnt-specific or Frizzled-specific nucleic acids or antibodies, hybridization probes and/or primers, and the like. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for inhibitors of Wnt signaling. Such kits can be prepared from readily available materials and reagents. For example, such kits can comprise one or more of the following materials: a Wnt or Frizzled polypeptide or polynucleotide, reaction tubes, and instructions for testing the desired Wnt signaling function (e.g., β catenin levels).

### Therapeutic Methods

### Administration of Inhibitors

The agents that inhibit Wnt signaling (e.g., antibodies) can be administered by a variety of methods including, but not limited to parenteral (e.g., intravenous, intramuscular, intradermal, intraperitoneal, and subcutaneous routes), topical, oral, local, or transdermal administration. These methods can be used for prophylactic and/or therapeutic treatment.

As noted above, inhibitors of the invention can be used to treat cancers associated with Wnt signaling. The compositions for administration will commonly comprise a inhibitor dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions containing inhibitors of the invention (e.g., antibodies) can be administered for therapeutic or prophylactic treatments. In therapeutic applications, compositions are administered to a patient suffering from a disease (e.g., breast cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of an inhibitor that is capable of preventing or slowing the development of cancer in a patient is referred to as a “prophylactically effective dose.” The particular dose required for a prophylactic treatment will depend upon the medical condition and history of the patient, the particular cancer being prevented, as well as other factors such as age, weight, gender, administration route, efficiency, etc. Such prophylactic treatments may be used, e.g., in a patient who has previously had cancer to prevent a recurrence of the cancer, or in a patient who is suspected of having a significant likelihood of developing cancer.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

Other known cancer therapies can be used in combination with the methods of the invention. For example, inhibitors of Wnt signaling may also be used to target or sensitize the cell to other cancer therapeutic agents such as 5FU, vinblastine, actinomycin D, cisplatin, methotrexate, and the like. In other embodiments, the methods of the invention can be used with radiation therapy and the like.

In some instances the antibody belongs to a sub-type that activates serum complement when complexed with the transmembrane protein thereby mediating cytotoxicity or antigen-dependent cytotoxicity (ADCC). Thus, cancer can be treated by administering to a patient antibodies directed against Frizzled proteins on the surface of cancer cells. Antibody-labeling may activate a co-toxin, localize a toxin payload, or otherwise provide means to locally ablate cells. In these embodiments, the antibody is conjugated to an effector moiety. The effector moiety can be any number of molecules, including labeling moieties such as radioactive labels or fluorescent labels, or can be a therapeutic moiety, such as a cytotoxic agent.

### Use of Wnt or Frizzled Polypeptides as Vaccines

In addition to administration of inhibitors of wnt signalling, the Wnt or Frizzled proteins or immunogenic fragments of them can be administered as vaccine compositions to stimulate HTL, CTL, and antibody responses against the endogenous proteins. Such vaccine compositions can include, e.g., lipidated peptides (see, e.g., Vitiello, et al. (1995) J. Clin. Invest. 95:341-349), peptide compositions encapsulated in poly(D,L-lactide-co-glycolide, “PLG”) microspheres (see, e.g., Eldridge, et al. (1991) Molec. Immunol. 28:287-294; Alonso, et al. (1994) Vaccine 12:299-306; Jones, et al. (1995) Vaccine 13:675-681), peptide compositions contained in immune stimulating complexes (ISCOMS; see, e.g., Takahashi, et al. (1990) Nature 344:873-875; Hu, et al. (1998) Clin. Exp. Immunol. 113:235-243), multiple antigen peptide systems (MAPs; see, e.g., Tam (1988) Proc. Nat'l Acad. Sci. USA 85:5409-5413; Tam (1996) J. Immunol. Methods 196:17-32); viral delivery vectors (Perkus, et al., p. 379, in Kaufmann (ed. 1996) Concepts in Vaccine Development de Gruyter; Chakrabarti, et al. (1986) Nature 320:535-537; Hu, et al. (1986) Nature 320:537-540; Kieny, et al. (1986) AIDS Bio/Technology 4:790-795; Top, et al. (1971) J. Infect. Dis. 124:148-154; Chanda, et al. (1990) Virology 175:535-547), particles of viral or synthetic origin (see, e.g., Kofler, et al. (1996) J. Immunol. Methods 192:25-35; Eldridge, et al. (1993) Sem. Hematol. 30:16-24; Falo, et al. (1995) Nature Med. 7:649-653).

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis, or Mycobacterium tuberculosis derived proteins. Certain adjuvants are commercially available as, e.g., Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding the Wnt or Frizzled polypeptides, or a fragment thereof, is administered to a patient. See, e.g., Wolff et. al. (1990) Science 247:1465-1468; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

Methods for the use of genes as DNA vaccines are well known, and include placing the desired gene or portion thereof under the control of a regulatable promoter or a tissue-specific promoter for expression in the patient. The gene used for DNA vaccines can encode full-length Wnt or Frizzled protein, or may encode portions of the proteins.

In a some embodiments, the DNA vaccines include a gene encoding an adjuvant molecule with the DNA vaccine. Such adjuvant molecules include cytokines that increase the immunogenic response to the polypeptide encoded by the DNA vaccine.

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, e.g., as a vector to express nucleotide sequences that encode Wnt or Frizzled polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover, et al. (1991) Nature 351:456-460. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata, et al. (2000) Mol. Med. Today 6:66-71; Shedlock, et al. (2000) J. Leukoc. Biol. 68:793-806; and Hipp, et al. (2000) In Vivo 14:571-85.

### Examples

The following examples are offered to illustrate, but not to limit the claimed invention.

### Materials and Methods

Cell lines

Human non-small-cell lung cancer (NSCLC) cell lines (NCI-H460, NCI-H838 and NCI-A549), normal lung cell line (CCL-75, fibroblast), human breast cancer cell lines (MCF-7 and SKBR-3), human colon cancer cell line SW480, and human mesothelioma cancer cell lines H28 were obtained from American Type Culture Collections (ATCC) (Manassas, Va.). Other human mesothelioma cancer cell line NCI-H290 was obtained from NIH (Frederick, Md.) and REN was kindly provided by Dr. Steven Albelda's lab at the University of Pennsylvania (Philadelphia, Pa.). Normal mesothelial cell line LP-9 was obtained from the Cell Culture Core Facility at Harvard University (Boston, Mass.). Human osteosarcoma cancer cell line Saos-2 was obtained from the Cell Culture Facility at UCSF. Mouse mammary cell lines: C57MG transfected with empty-vector (C57MG) and transfected with Wnt-1 (C57Wnt-1) were kindly provided by Dr. Frank McCormick's Lab at UCSF Cancer Center. These cells, except CCL-75, LP-9, and Saos-2, were cultured in RPMI 1640 supplemented with 10% foetal bovine serum, penicillin (100 IU/ml) and streptomycin (100 μg/ml). CCL-75 was cultured in MEM with Earle's BSS containing 2 mM L-glutamine, 1.0 mM sodium pyruvate, 0.1 mM nonessential amino acids, 1.5g/L sodium bicarbonate and 10% foetal bovine serum. LP-9 was cultured in M199 containing 15% CS plus 10 ng/ml of EGF plus 0.4 μg/ml of HC. Saos-2 was cultured in McCoy's 5a medium supplemented with 2 mM L-Glutamine and 15% foetal bovine serum. Normal human small airway epithelial cells (SAEC) and bronchial epithelial cells (BEC) were obtained from Clonetics (Walkersville, Md.) and cultured in Clonetics SAGM™ Bullet Kit. All cells were cultured at 37° C. in a humid incubator with 5% CO₂.

Antibody Incubation with Cells

Cells were plated in 6-well plates one day before experiments. Then normal media were replaced by media containing antibodies at various concentrations and the cells were incubated at 37° C. in a humid incubator with 5% CO₂. At various time points the cells were collected using standard protocols for further analysis. Purified anti-Wnt-1 and anti-Wnt-2 polyclonal antibodies (IgG from goat) were obtained from Santa Cruz Biotechnology (Santa Cruz, Calif.). As a control, purified anti-SOCS-3 (SOCS-3 is a cytoplasmic protein) polyclonal antibody (IgG from goat) (also from Santa Cruz Biotechnology (Santa Cruz, Calif.)) was used in parallel experiments.

### Western Blotting

Standard protocol as described previously (Yoshikawa et al., Nat Genet, 28:29-35 (2001)) was used. Anti-Dvl3, anti-survivin, and anti-Bcl-2 antibodies were obtained from Santa Cruz Biotechnology (Santa Cruz, Calif.). Anti-caspase3, anti-caspase9 antibodies were from Oncogene (Cambridge, Mass.). Anti-β-actin, anti-Smac/Diablo and anti-β-catenin antibodies were obtained from Cell Signaling Technology, Inc. (Beverly, Mass.). Anti-cytochrome c antibody was obtained from BD Biosciences. Anti-Active®-JNK antibody was obtained from Promega (Madison, Wis.). For detecting alteration of β-catenin cytosolic extracts were prepared and examined as described previously (Wang et al., Mol Cell Biol, 19:5923-5929 (1999)).

### Apoptosis Analysis

Cells were harvested by trypsinization and stained using an Annexin V FITC Apoptosis Detection Kit (Oncogene, Cambridge, Mass.), according to the manufacture's protocol. Then stained cells were immediately analyzed by flow cytometry (FACScan; Decton Dickinson, Franklin Lake, N.J.). Early apoptotic cells with exposed phosphatidylserine but intact cell membranes bound to Annexin V-FITC but excluded propidium iodide. Cells in necrotic or late apoptotic stages were labeled with both Annexin V-FITC and propidium iodide.

### RNA Interference Analysis

Cells were plated into a 6-well plate with fresh medium without antibiotics 24 hrs before experiments. The ion-exchange HPLC-purified siRNAs (Wnt-1 siRNA and nonsilencing siRNA control, >97% pure) were purchased from Qiagen-Xeragon (Germantown, Md.). The lyophilized siRNAs were dissolved in annealing buffer and reheated to 95° C. for 1 min followed by 1 hr at 37 ° C. incubation. The siRNA analysis was performed as previously described protocol (Elbashir, et al., Methods 26, 199-213, 2002) with some modifications. After siRNA transfection, plates were incubated for 3-5 days at 37° C. before further analysis.

In vivo Tumor Suppression Study

Human NSCLC cell line H460 and human breast cancer cell line MCF-7 were cultured as described in previous section. Female nude mice, 5-10 weeks old, were injected with 4×10⁶ tumor cells in the dorsal area in a volume of 100 μl. Animals were then intraperitioneally injected with monoclonal anti-Wnt-1 antibody, a control monoclonal antibody, or PBS buffer in a volume of 100 μl as well. Both the monoclonal anti-Wnt-1 antibody and the control monoclonal antibody were injected at the dose of 50 μg. Each injection was done once weekly. Each group consisted of 5 mice. Tumor size was determined at weekly intervals according to standard techniques.

### Statistical Analysis

Data shown represent mean values (±S.E.M.). Unpaired T-Test in the Excel was used for comparing different treatments and cell lines.

### Results

### Example 1

### Anti-Wnt Antibody Specifically Induces Apoptosis in a Number of Different Human Cancer Cells

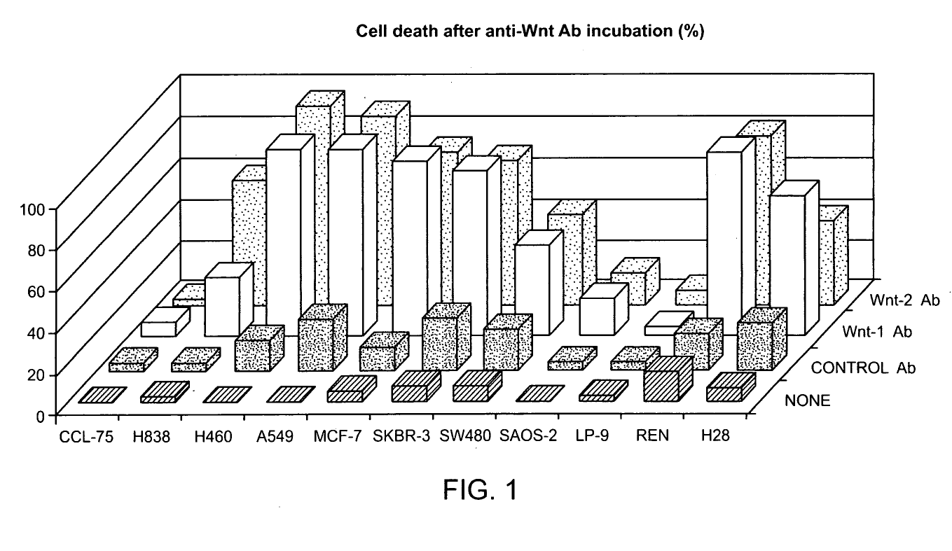

We examined whether neutralizing Wnt signaling by using anti-Wnt antibodies could inhibit cell survival in these cancers. When we incubated a number of cancer cell lines with either anti-Wnt-1 or Wnt-2 antibody (at 10 μg/ml) for about 32 hrs (we examined three non-small-cell lung cancer (NSCLC), two breast cancer, two colorectal cancer, one sarcoma, and two mesothelioma cell lines), we found that both antibodies could cause significant cell death (from 30% to 97%), except for one colorectal cancer cell line SW480 (only 4-8%)(FIG. 1). In contrast, an antibody against a cytoplasmic protein (SOCS3) (at 10 μg/ml) did not show dramatic cytotoxicity in most of those cell lines (from 4% to 45%)(FIG. 1). Interestingly, none of the antibodies had dramatic effect on the two normal cell lines that we examined (one was normal lung fibroblast (CCL-75) and the other was normal mesothelial cell line (LP-9)) (from 2% to 8%)(FIG. 1).

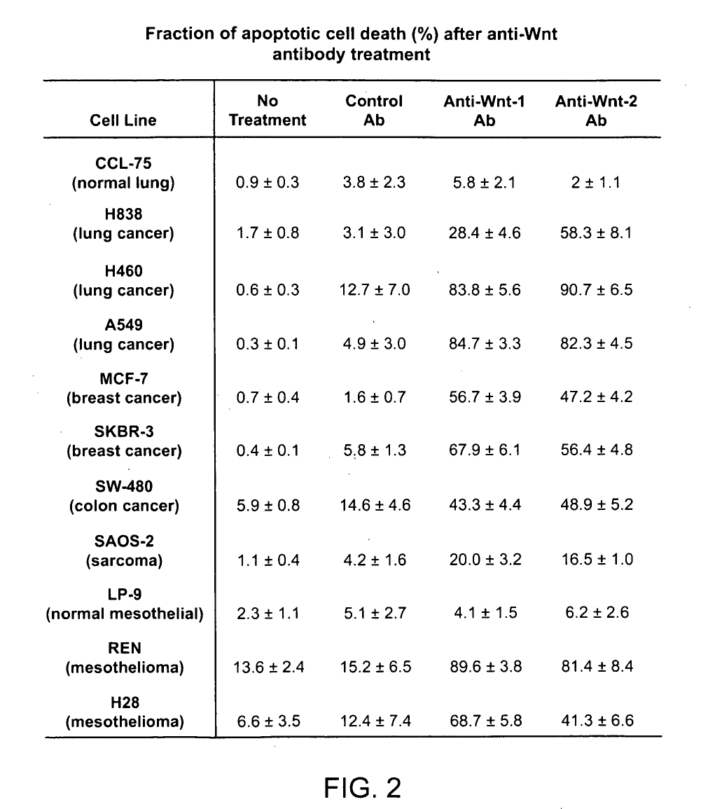

To determine whether anti-Wnt antibody mediated cell death was due to modification of apoptosis, the cells were stained with Annexin V-FITC and propidium iodide (PI) after antibody treatment for about 32 hrs, followed by apoptosis analysis using flow cytometry. As shown in FIG. 2, we found that in the cancer cell lines we examined majority of cell death was via apoptosis (from 28% to 91%). Again, apoptosis was not detected in the two normal cell lines after the antibody incubation (only 2% to 6%)(FIG. 2). These results demonstrated that blocking Wnt signaling by using anti-Wnt antibody could specifically induce apoptosis in cancer cells, but not in normal cells.

### Example 2

### Anti-Wnt Antibody-Induced Apoptosis is Correlated with the Wnt Expression

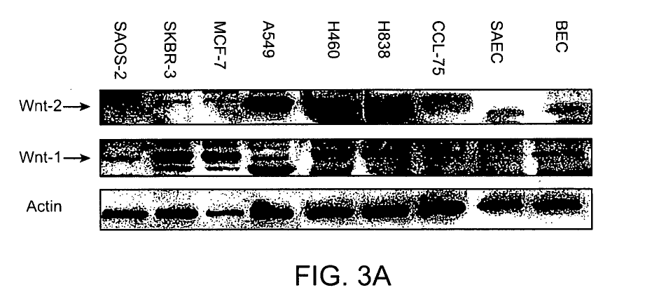

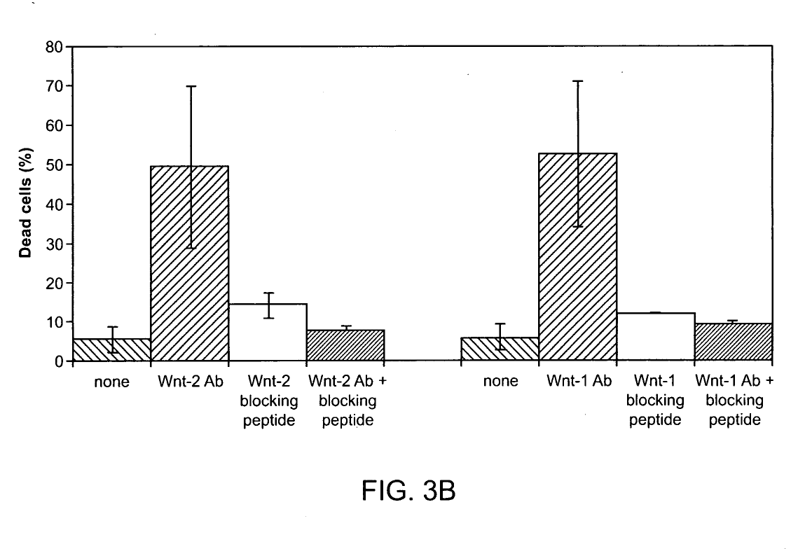

To investigate whether anti-Wnt antibody-induced apoptotic effect was associated with status of the Wnt proteins, we examined e Wnt expression in the cell lines we tested. As shown in FIG. 3A, we found that Wnt-1 had high-level expression in the cancer cell lines that were sensitive to anti-Wnt-1 antibody treatments. However, in the normal lung cell line CCL-75 that was not sensitive to the antibody treatment (see FIG. 1) only minimal Wnt-1 and expression was detected. No Wnt-1 expression was detected in two primary normal lung cells (small airway epithelial cells (SAEC) and bronchial epithelial cells (BEC)) (FIG. 3) and in normal mesothelial cell line (LP-9) (data not shown). Similar observations were made regarding Wnt-2 expression.

As a control, we examined apoptosis induction of co-incubation of anti-Wnt antibody and blocking peptide for anti-Wnt antibody in an NSCLC cell line. After about 24 hr incubation we found that anti-Wnt antibody induced apoptosis could be inhibited by its blocking peptide significantly (P<0.01). Taken together, these results indicated that anti-Wnt antibody-induced apoptosis was correlated with the Wnt expression in the cells we examined.

### Example 3

### Anti-Wnt-1 Antibody-Induced Apoptosis is a Fast Process and Dose Dependent

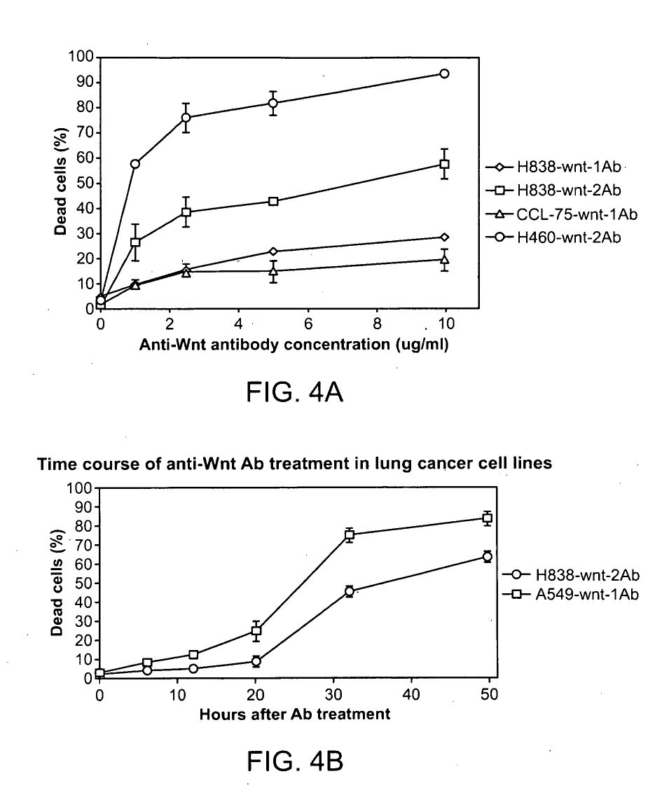

We performed dosage and time course experiments on two NSCLC cell lines: H838 and A549 (FIG. 4A and FIG. 4B). Flow cytometry analysis after about 32 hr incubation of anti-Wnt antibody showed that 1 μg/ml antibody could induce apoptosis. A concentration of 20 μg/ml of either antibody caused dramatic apoptotic cell death. Anti-Wnt-1 antibody (at concentration of 8 μg/ml) induced apoptosis could be detected as early as after 6 hr incubation and after 50 hr incubation almost all cells were found undergoing apoptosis or necrosis. In contrast, control anti-SOCS3 antibody did not have effect on those cancer cells in the parallel experiments. Anti-Wnt-1 antibody incubation with normal lung cell line (CCL-75) was also insensitive to either time or dosage.

### Example 4

### Anti-Wnt Antibody-Induced Apoptosis is Associated with Down-Regulation of Dvl-3 and Cytosolic β-Catenin

Wnt signaling has been shown to activate β-catenin/Tcf-mediated transcription through Dvl. Wnt signaling also stabilizes cytosolic β-catenin. Thus, we determined whether anti-Wnt antibody induced apoptosis was dependent on Dvl and destabilization of cytosolic β-catenin. We found that both Dvl and cytosolic β-catenin level was dramatically down regulated after anti-Wnt antibody treatment in the cancer cells we examined. In contrast, no change of both Dvl and cytosolic β-catenin level was found in the normal cell line after anti-Wnt antibody treatment. We also detected apoptosis after we treated cancer cells with Apigenin that blocks CK-1 activity, which in turn inhibits Dvl activity. The cytosolic β-catenin level was downregulated by Apigenin treatment. These results suggested that anti-Wnt antibody induced apoptosis was, at least in part, through inhibiting the function of Dvl/β-catenin, the downstream components of the Wnt/Frizzled signaling pathway.

### Example 5

### Anti-Wnt Antibody Induces Apoptosis through Down-Regulation of Survivin Expression and Subsequent Activation of Caspase-3

Next, we examined the molecular mechanism of this specific anti-Wnt antibody-induced apoptosis in cancer cells. It has been found that activating caspase-9 switches on apoptotic pathway and activated caspase-9 amplifies the apoptotic pathway by cleaving and activating down stream executive caspases, such as caspase-3. Survivin (one of the apoptosis inhibitor IAP family members) plays an important role in inhibiting activation of both caspase-3 and caspase-9. In cancer cells that were sensitive to anti-Wnt antibody treatment both cleaved (active) form of caspase-9 and caspase-3 were up regulated. We also found that survivin expression was significantly down regulated in these cancer cells. In contrast, in the normal cell line CCL-75 that was not sensitive to anti-Wnt antibody treatment we did not detect up regulation of cleaved form of both caspases and down regulation of survivin expression. These results demonstrated that anti-Wnt antibody induced apoptosis by inhibiting apoptosis inhibitor-survivin and activating of caspase-9 and caspase-3.

### Example 6

### Anti-Wnt Antibody-Induced Apoptosis is Associated with Releasing of Smac/Diablo and Cytochrome C from Mitochondria to the Cytosol and JNK Activation

During apoptosis, Smac/Diablo (second mitochondria-derived activator of caspase/direct IAP-binding protein with low pI) functions to remove the IAP-mediated caspase inhibition. Stimulation of apoptosis causes releasing of Smac/Diablo from the intermembrane space of mitochondria into the cytosol, together with cytochrome c. Cytochrome c directly activates Apaf-1 and caspase-9 and Smac/Diablo interacts with multiple IAPs to remove IAP-mediated inhibition of both initiator and effector caspases. Consistent with above results where caspase-3 activity increases in the cancer cells, but not in the normal cells, we found increase level of both Smac/Diablo and cytochrome c in the cytosol of the cancer cells after anti-Wnt antibody treatment, but not in that of the normal cells. Our results indicate that both Smac/Diablo and cytochrome c are likely involved in this anti-Wnt antibody induced apoptosis by removing survivin and/or other IAPs-mediated inhibition and direct activation of caspases, respectively.

To further determine how this specific anti-Wnt antibody-induced apoptosis is regulated, we examined other components in the apoptotic pathway. Surprisingly, we found that JNK activity was dramatically increased in the cancer cells after the treatments. In contrast, in the normal cell line CCL-75 that was not sensitive to anti-Wnt antibody treatment increase of JNK activity was not detected. We also found that over-expression of Dvl in a normal mesothelial cell line down regulated JNK activities. In addition, inhibition of Dvl by using Apigenin to block CK-1 activity could also increase JNK activity. Taken together, the anti-Wnt antibody-induced apoptosis involves JNK activation and increase of the JNK activity after blocking Wnt signaling is likely through inactivating Dvl.

### Example 7

### Anti-Wnt-1 Antibody Specifically Induces Apoptosis in Wnt-1 Transfected Mouse Mammary Cells

As one control, we compared apoptotic effect induced by anti-Wnt-1 antibody incubation in mouse C57MG versus Wnt-1-transfected C57MG cells, because these cells have already been characterized for their free pool of β-catenin. It has been shown that Wnt-1 signaling is on in Wnt-1-transfected C57MG cells, but off in un-transfected or empty-vector-transfected C57GM cells. Flow cytometry analysis after 42 hr anti-Wnt-1 antibody incubation showed no noticeable effect in un-transfected or empty-vector-transfected C57GM cells (less than 10% cell death after incubation). However, significant cell death was seen in Wnt-1-transfected C57MG cells (over 85% cell death, P<0.001).

The anti-Wnt-1-induced apoptosis in Wnt-1-transfected C57MG cells also appears to be linked with down-regulation of Dvl-3 and cytosolic β-catenin, and through down-regulation of survivin expression and subsequent activation of caspase-3, and through releasing of Smac/Diablo and cytochrome c from mitochondria to the cytosol and JNK activation. The Wnt-1-transfected C57MG cell line serves as an ideal control model for our discovery, and these data provided more support to our finding in human cancer cells.

### Example 8

### An Anti-Wnt-1 Monoclonal Antibody Shows Induction of Apoptosis in Different Human Cancer Cells In vitro and Suppresses Tumor Growth In vivo

Antibodies were raised against peptides derived from human Wnt-1. In particular, hybridoma cell liens were generated using SEQ ID NO:2 and SEQ ID NO:4. One of the monoclonal antibodies was raised against a synthetic peptide corresponding to amino acid 201-212 of the human Wnt-1 (Ac-HNNEAGRTTVFS-amide). The antibody was affinity purified using Protein A. Wnt-1 expression in numerous human cell lines was evaluated using this monoclonal antibody. The cell lines included three breast cancer cell lines (HuL100, MCF-7, and SKBR-3), five malignant plural mesothelioma cell lines (REN, H513, H290, MS-1, and H28), four non-small-cell lung cancer (NSCLC) cell lines (A549, H460, H838, and H1703), two sarcoma cell lines (MES-SA and Saos-2), one colon cancer cell line SW480, and four normal cells (small airway epithelial cells (SAEC) and normal human bronchial epithelial cells (NHBE), LP-9, and CCL-75). We found higher-level Wnt-1 expression in most of these cancer cell lines, except for A549, MES-SA, H513, SKBR-3 and SW480, which had no or minimal Wnt-1 expression. No Wnt-1 expression was observed in the two primary normal lung cells (SAEC and NHBE). We only detected minimal Wnt-1 expression in the normal lung fibroblast CCL-75 and in a normal mesothelial cell line (LP-9). As a control experiment, we found Wnt-1 expression using the same monoclonal antibody in Wnt-1-transfected mouse mammary cells (C57Wnt-1), but not in empty-vector-transfectcd cells (C57mv7).

To test if the anti-Wnt-1 monoclonal antibody can specifically bind to the native form of Wnt-1 protein in cultured cells, we performed immunoprecipitation using monoclonal antibody alone or monoclonal antibody blocked by pre-incubation with blocking peptide (30-fold over the antibody) in cell extracts from several cell lines. C57Wnt-1 and C57mv7 cells served as positive and negative controls, respectively. NSCLC (H460) and breast cancer (MCF-7) cell lines were also tested. In C57Wnt-1, H460 and MCF-7 cells Wnt-1 protein was precipitated by the anti-Wnt-1 monoclonal antibody. In contrast, when the anti-Wnt-1 monoclonal antibody was preincubated with blocking peptide, its ability to precipitate Wnt-1 protein was blocked in these cells. No Wnt-1 protein was precipitated by either anti-Wnt-1 monoclonal antibody alone or monoclonal antibody pre-incubated with blocking peptide in the negative control. These data indicate that the anti-Wnt-1 monoclonal antibody specifically binds to native form of Wnt-1 protein.

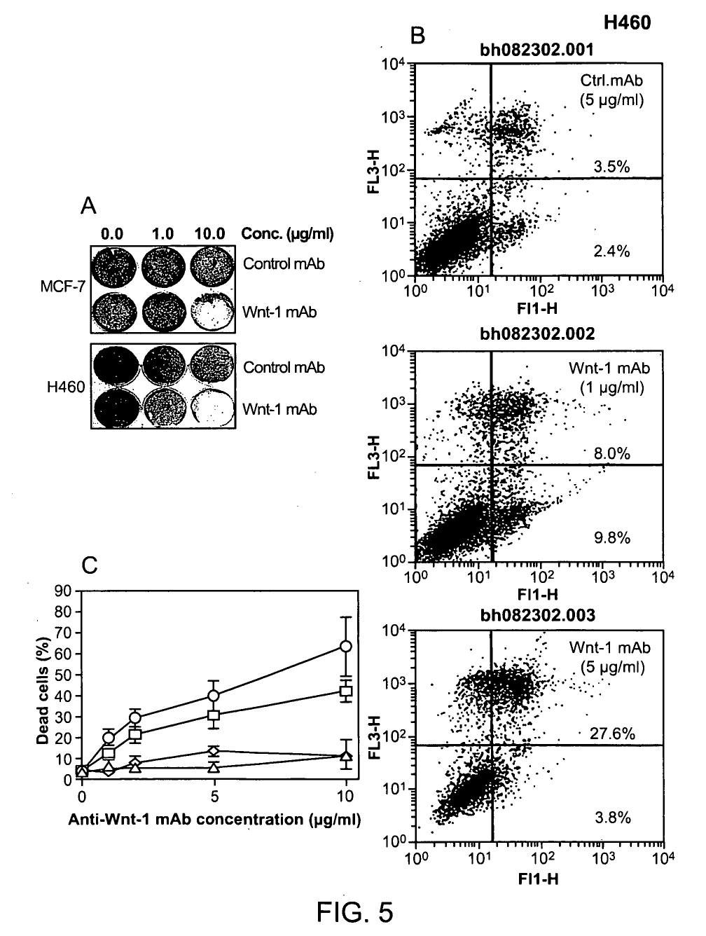

Next, we treated a NSCLC cell line H460 and a breast cancer cell line MCF-7 with this monoclonal antibody. After about 48-72 hrs of incubation we found significant cell death in both cell lines (over 60% cell death at 10 μg/ml of the antibody, P<0.001) (FIG. 5a). We saw no noticeable effect, however, in both cell lines after control monoclonal antibody treatment. Cell killing was largely due to induction of apoptosis (FIG. 5b). Induction of apoptosis by this monoclonal antibody was dosage and time dependent (over 60% cell death in H460 at 10 μg/ml of the antibody after about 72 hrs of incubation and over 40% cell death in MCF-7 at 10 μg/ml of the antibody after about 48 hrs of incubation) (FIG. 5c). We also treated other cancer cell lines that have Wnt-1 overexpression, including breast cancer HuL100, NSCLC H1703, mesothelioma H28 and REN, and sarcoma Saos-2. We found similar results.

As a specificity control, we examined induction of apoptosis by using monoclonal antibody blocked by overnight pre-incubation with blocking peptide (30-fold over the antibody) in H460, MCF-7 and H1703. After 48 hrs of incubation, we found that anti-Wnt-1 antibody-induced apoptosis could be inhibited significantly by its blocking peptide (P<0.003). Same dose blocking peptide alone did not affect viability of these cells (8.0 μg/ml for 48 hrs). As a negative control, we used A549 cells that lack significant Wnt-1 expression. After about 48 hr treatment with either monoclonal antibody alone (8.0 μg/ml) or with monoclonal antibody blocked by preincubation with blocking peptide (30-fold over the antibody), no significant induction of apoptosis was detected. This result is consistent with Wnt-1 expression status of A549 cells.

The Anti-Wnt-1 Monoclonal Antibody Inhibits Wnt/β-Catenin Signaling Pathway and Induces Apoptosis through Release of Cytochrome c, Down-Regulation of Survivin Expression and Subsequent Activation of Caspase-3

We found that both Dvl-3 and cytosolic β-catenin as well as Cyclin D1 levels were down-regulated after anti-Wnt-1 monoclonal antibody treatment in the cancer cells examined. We also performed TOP/FOP assay in these cells and found that TCF dependent transcriptional activity decreased after the monoclonal antibody treatment. In contrast, no change of either Dvl, cytosolic β-Catenin levels or TCF dependent transcriptional activity was found in normal cells or cancer cells lacking (or with minimal) Wnt-1 expression after anti-Wnt-1 monoclonal antibody treatment. These results suggest that anti-Wnt-1 monoclonal antibody induced apoptosis is mediated, at least in part, through inhibiting Dvl/β-catenin dependent transcription.

In H460 cells in which anti-Wnt-1 monoclonal antibody induces apoptosis, we found that cleaved (active) form of caspase-3 was up-regulated. Consistent with the caspase-3 activity, we detected increased level of Cytochrome c in the cytosol of H460 cells after anti-Wnt-1 monoclonal antibody treatment. In addition, we found that Survivin expression was down-regulated in these H460 cells after the antibody treatment.

Others have shown that Wnt-l signaling is on in C57Wnt-1 cells, but off in C57mv7 cells 11. As a control, we tested if anti-Wnt-1 monoclonal antibody could inhibit Wnt/β-catenin signaling in C57Wnt-1 cells. Western analysis C57mv7 showed that both cytosolic β-catenin and Cyclin D1 levels were down-regulated after anti-Wnt-1 monoclonal antibody treatment (8.0 μg/ml for 48 hrs) in C57Wnt-1 cells, but no Cyclin D1 expression was detected in C57mv7 cells. Cytosolic β-catenin level in C57mv7 cells also remained unchanged after anti-Wnt-1 monoclonal antibody treatment. Consistently, TCF-dependent transcriptional activity measured by TOP/FOP assay also decreased in C57Wnt-1 cells, but remained unchanged in C57mv7 cells. These data indicate that the anti-Wnt-1 monoclonal antibody inhibits Wnt/β-catenin signaling in the cell lines examined.

### RNA Interference

We followed the protocol described by Elbashir et al. (Elbashir, et al., Methods 26, 199-213, 2002) to investigate the effect of silencing Wnt-1 expression by using RNAi. Similar to the monoclonal anti-Wnt-1 antibody, treatment with Wnt-1 siRNA for 3-5 days induced apoptosis in cancer cell lines, e.g., MCF-7 cells, that express Wnt-1. Significant apoptosis was induced at 100 nM Wnt-1 siRNA, but no apoptosis was induced by either non-silencing siRNA control (100 nM) or transfection reagents. We confirmed the silencing of Wnt-1 expression after Wnt-1 siRNA treatments (100 nM for 72 hrs) by Western analysis (non-silencing siRNA served as control (100 nM for 72 hrs)). To determine whether the apoptotic effects correlated with the inhibition of Wnt-1 signaling, we also showed that expression levels of Dvl-3, cytosolic β-catenin, and Survivin were down-regulated after Wnt-1 siRNA treatment.

### Inhibition of Cancer Growth In Vivo

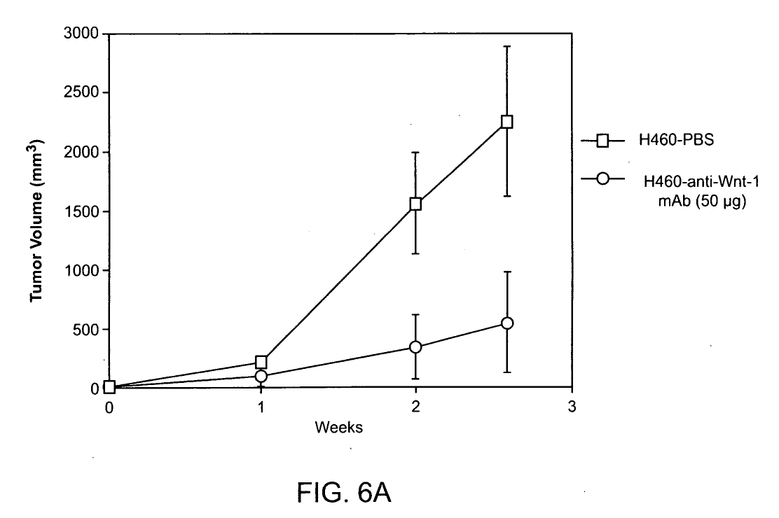

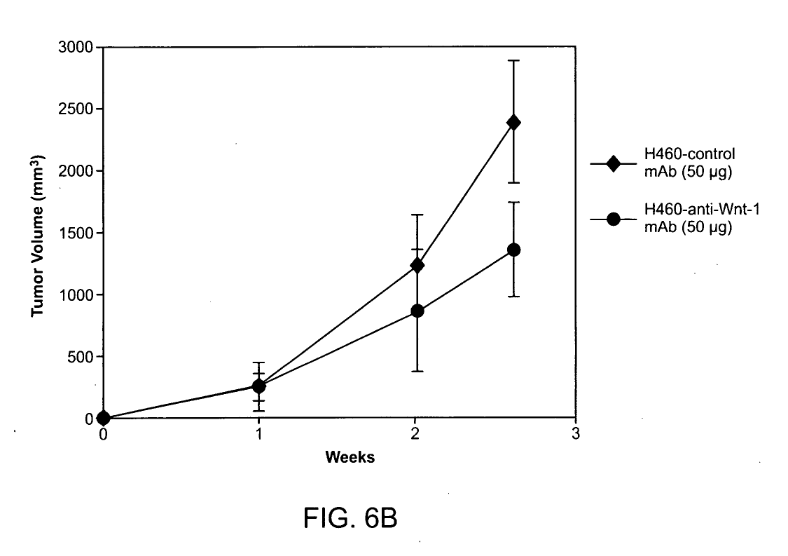

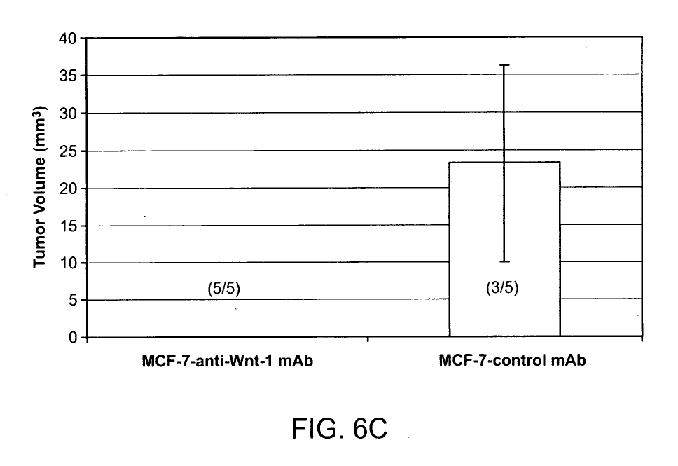

Next we tested whether the monoclonal anti-Wnt-1 antibody could suppress tumor growth in vivo. We injected H460 and MCF-7 cells into nude mice, respectively. Animals were then received 50 μg of the monoclonal anti-Wnt-1 antibody, a control monoclonal antibody or PBS via intraperitoneal (i.p.) injection once weekly. FIG. 6A shows although the control antibody had no appreciable suppression, the monoclonal anti-Wnt-1 antibody at such dose significantly inhibited growth of both tumor types (P<0.001). Suppression of the tumor growth was seen not only when the monoclonal anti-Wnt-1 antibody injection was started immediately after tumor cell inoculation (FIG. 6A), but also when the treatment was initiated after the tumors were already established (one week after tumor cell inoculation) (P<0.005) (FIG. 6B). In the studies using MCF-7 cells (FIG. 6C), tumor volume is whons after 3 weeks treatment with anti-Wnt-1 monoclonal antibody and control monoclonal antibody. Five animals are in each group. None of the animals that were treated with anti-Wnt-1 mAb injections developed tumors. However, three of five control animals developed tumors. (I.P. injections were administered once weekly one week after MCF-7 cell inoculation.).

The sequences of the V_{H} and V_{L} regions of the anti-Wnt-1 monoclonal antibody used in the studies described above were determined. The CDR and framework (FR) regions were amplified from the hybridoma cell lines by RT-PCR and analyzed by agarose gel. The sequences of the V_{H} and V_{L} regions are shown in FIG. 7.

### Example 9

### Wnt-2 Expression and Wnt-2 Monoclonal Antibody-Induced Apoptosis

Wnt-2 gene expression was analyzed in multiple human cancer and matched non-cancerous tissue specimens. Radiolabeled Wnt-2 cDNA probes were hybridized with the Cancer Profiling Array II (BD Biosciences, Inc.), which contains 19 different types of human tumors with matched non-cancerous tissue specimens. Wnt-2 was overexpressed in the majority of colon, stomach, rectal, and thyroid tumors in comparison with their normal counterparts.

A monoclonal antibody was raised against a synthetic peptide corresponding to amino acids 49-63 (SSQRQLCHRHPDVMR) of human Wnt-2. The antibody was affinity purified using Protein A. The effect of Wnt-2 monoclonal antibodies on apoptosis was determined in human melanoma FEMX and LOX cells. The results show that the anti-Wnt-2 monoclonal antibody induced apoptosis in FEMX and LOX human melanoma cells. The antibody also induced apoptosis in human colon cancer HCT-116 and SW480 cells, as did the anti-Wnt-1 monoclonal antibody of Example 8.

The sequences of the V_{H} and VL regions of the anti-Wnt-1 monoclonal antibody used in the studies described above were determined. The CDR and framework (FR) regions were amplified from the hybridoma cell lines by RT-PCR and analyzed by agarose gel. The sequences of the V_{H} and V_{L} regions are shown in FIG. 7.

### Example 10

### Mesotheliomas have over Expression of β Catenin through Activation of Dvl, and Transcriptional Activity of β Catenin is Correlated to Tumorigenecity

We further investigated the role-of wnt signaling in mesotheliomas. We found that. most mesothelioma cells overexpress Dvl-3. Expression of Dvl-3 and cytosolic β-Catenin was investigated in mesothelioma cells using western blots. Western blot analysis showed that 8 of 10 fresh malignant mesothelioma tissues overexpress Dvl-3 protein and have increased cytosolic β-catenin compared with autologous matched normal pleural tissue controls. Furthermore, five additional malignant mesothelioma cells tested (two primary malignant pleural mesothelioma cultured cells and three cell lines, LRK1A, REN, and H513) had high levels of Dvl-3 and cytosolic β-catenin, compared with normal pleuralbcontrols. Immunohistochemical analysis of several of the tumor cells demonstrated cytoplasmic, nuclear, and membrane bound β-catenin. We found no mutation in exon 3 of β-catenin in 13 mesothelioma tissues, including the cases tested by Western blot and two malignant effusions. Exon 3 was selected for mutational analysis because it encodes the NH2-terminal regulatory domain of β-catenin, which was previously found to contain activating mutations. Furthermore, we detected no mutation in the complete coding region of β-catenin in three mesothelioma cell lines (LRK1A, REN, and H513).

Transcription activity of β-catenin using a Tcf-dependent luciferase reporter gene was also examined. Western blot analysis was used to confirm APC, GSK-3β, and Tcf4 expression in all tumors under studied. Transcriptional activity mediated by Tcf-β-catenin protein complexes was assayed as a ratio to reporter gene activity in mesothelioma cell lines with significant overexpression of Dvl and cytosolic β-catenin. Cells were transiently transfected with either the pTOPFLASH or pFOPFLASH reporter construct, which contained multimerized wild type or mutant Tcf-binding motifs upstream of the Firefly Luciferase cDNA with the pRL-TK internal control reporter construct that contains the Renilla Luciferase cDNA. Tcf-mediated gene transcription was determined by the ratio of pTOPFLSH:pFOPFLLASH luciferase activity after 24 h, each corrected for luciferase activities of the pRL-TK reporter.

Mesothelioma cells with high levels of cytosolic β-catenin, including cells from malignant pleural mesothelioma effusions, LRK1A, REN, and H513 cell lines showed a significant fold increase (1.5-2.4-fold, P<0.01) in Tcf-mediated gene transcriptional activity of β-catenin (pTOPFLASH/pFOPFLASH). In contrast, normal mesothelial cells, which have minimal expression of cytosolic β-catenin, showed no difference.

Transcriptional activity of β-catenin in Tcf β-catenin mediated reporter assay, which was confirmed by the reporter assay under expression of Gal4-β-catenin fusion protein, categorized mesothelioma cells, which was positive transcriptional activity or negative. In mesothelioma cells, the cytosolic expression of β-catenin was inhibited by adding apigenin, which can degradate Dvl through corruption of casein kinase II, and PDZ-Dvl, but was enhanced by wild type Dvl. Furthermore, PDZ-Dvl inhibited the Tcf dependent transcriptional activity. Stably expression of PDZ-DVL inhibited the colony formation in the mesothelioma cells, which had the positive transcriptional activity of β-catenin, but the mesothelioma cells, which had negative transcriptional activity of β-catenin, showed satble colony formation. Our confirmation of Tcf dependent transcription and Gal4-β-catenin fusion protein was well correlated to the results of the tumorigenesity in mesothelioma cell.

Dvl-3 stabilizes the cytosolic β-catenin in mesothelioma cells. We have confirmed that fresh malignant mesothelioma cells from pleural effusions demonstrated the overexpression of Dvl-3 protein with expression of the cytosolic β-catenin by Western blot analysis. Except for H28 cell line, which contains a homozygous deletion of β-catenin region, all other malignant cells tested with high expression of Dvl-3 showed remarkably higher expression of cytosolic β-catenin than cells from normal pleural tissue. These results demonstrate that activation of Dvl-3 translocate β-catenin from membrane to cytoplasm and nucleus in mesothelioma cells.

β-catenin activates the Tcf dependent transcription in mesothelioma cells. Transcriptional activation mediated by Tcf-β-catenin protein complexes was determined and compared by reporter gene analysis in mesothelioma cell lines with significant overexpression of Dvl and cytosolic β-catenin, and another mesothelioma cell line, H28, that lacks expression of β-catenin due to homozygous deletion but contains the expression of Dvl. Cells were transiently transfected with either the pTOPFLASH or pFOPFLASH reporter construct, which contained multimerized wild type or mutant Tcf-binding motifs upstream of the Firefly Luciferase cDNA with the pRL-TK internal control reporter construct that contains the Renilla Luciferase cDNA. Tcf-mediated gene transcription was determined by the ratio of pTOPFLSH:pFOPFLLASH luciferase activity after 24 h, each corrected for luciferase activities of the pRL-TK reporter. Of mesothelioma cells with high expression of cytosolic β-catenin, malignant effusion of a mesothelioma patient, LRK1A and REN showed 1.8-2.4 fold increase in transcriptional activity of the pTOPFLASH reporter, and H290 and H513 exhibited 1.4-1.5 fold increase. In contrast, H28 and normal mesothelial cells, which have no or slight expression of cytosolic β-catenin, showed no deference between the pTOPFLASH and pFOPFLASH activity. These results indicate that a great deal of β-catenin can be the transmitter in mesothelioma cells.

Gal4-β-catenin activates pG5 in mesothelioma cells. Furthermore, the control of transcriptional activity by β catenin in mesothelioma cells was measured using the GAL4-β-catenin construct to exclude the possibility that mesothelioma cell lines lack the necessary transcriptional machinery. After cotransfection of pSG424-GAL4-β-catenin, which transcribes the GAL4-β catenin fusion protein, and pG5, a CAT reporter construct, GAL4-β-catenin mediated gene transcription was determined. These activities were normalized to the CAT activity of the pG5 reporter construct only to exclude the background level of activation. Gal4-β-catenin protein was expressed in all transfected mesotheliomas by Western blot analysis using Flag antibody. LRK1A, REN and H28 cells showed 10-25-fold increased activity after co-transfection with pSG-GAL4-β-catenin and pG5 reporter construct as compared with control transfection of pG5. Hela cells exhibited a 25-fold increase. In contrast, H513 showed a few-fold increase, and H290 showed only background activity. These high activity confirm that the abundant β-catenin mesotheliomas is capable of transcriptional activity in LRK1A and REN, but it is impossible in H290, even though it has a higer activation in Tcf dependent transcription.

Apigenin induces the degradation of Dvl, which results in the stability of cytosolic β-catcnin. Apigenin promotes degradation of Dvl and β-catenin through inhibition of casein kinase II in mammary epithelial cells, leading to the inhibition of cell proliferation. Adding Apigenin to media inhibited the growth of LRK1A, REN and H290 over the course of a 48 hours treatment degradated Dvl and cytosolic β-catenin. These results suggest that the activation of Dvl by casein kinase II regulates, in part, the translocation of β-catenin in mesothelioma cells.

PDZ-Dvl inhibits the function of endogenous Dvl and the stability of cytosolic β-catenin in mesothelioma cells. Dishevelled proteins possess three conserved domains, a dix domain, present in the Wnt antagonizing protein Axin; a PDZ domain involved in protein-protein interactions, and a DEP domain found in proteins that regulate Rho GTPases. Function of three conserved domains is required for up-regulation of β-catenin and for stimulation of LEF-1-mediated transcription in mammalian cells. Transfection of pCS-mouse Dvl-1 to 293T cells resulted in a 15-fold increase in Tcf-mediated gene transcriptional activity of β-catenin, in accordance with other investigators' findings. This activity was inhibited by a pCS-mouse Dvl-1 construct by cotransfection of pCS-cDNA-encoding ΔPDZDvl-1. Furthermore, Tcf-dependent transcriptional activity of β-cateninbin LRK1A was reduced by transfection of pCS-ΔPDZ-Dvl-1 (from 2.1- to 1.3-fold, P<0.05), whereas transfection of pCS-Dvl-1 enhanced Tcf-dependent transcriptional activity of β-catenin (from 2.1- to 3.8-fold, P<0.05), indicating that β-catenin Tcf-mediatedbtranscription in these cells is regulated significantly by Dvl.b.

To examine additional Wnt pathway activation in malignant pleural mesothelioma, we transfected retrovirally, ΔPDZ-Dvl-1 and wild-type Dvl-1 into LRK1A, REN, and H513 cell lines, respectively. Retrovirus transfection of pLXN-ΔPDZ-Dvl-1 induced expression of ΔPDZ-Dvl-1 protein, which significantly reduced the expression of cytosolic β-catenin in all cells tested compared with controls (P<0.05). These results demonstrate that Dvl regulates cytosolic β-catenin in mesothelioma cells.

Using Atlas human cancer 1.2 array, c-myc expression in REN was shown to be down-regulated by ΔPDZ-Dvl-1 transfection. On the other hand, COX-2, which has been confirmed to be one of target genes of Wnt/β-catenin pathway, was down-regulated by ΔPDZ-Dvl-1 transfection using Western blot analysis.

### Transfection of ΔPDZ-Dvl Inhibits Tumorigenicity of Mesothelioma Cell Lines in Soft Agar and in Athymic Mice.

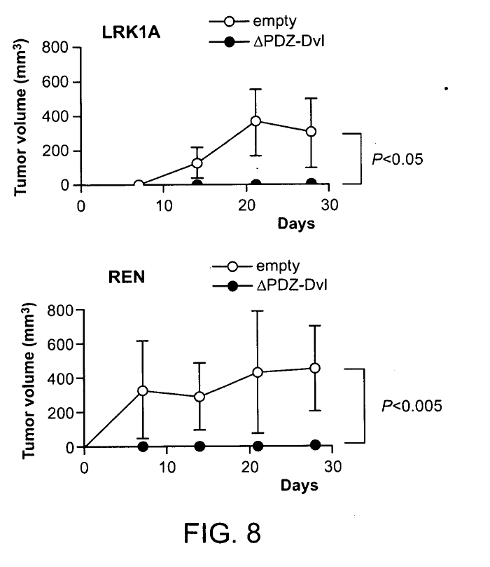

We examined the role of the Dvl/β-catenin pathway in relationship to cell growth in malignant pleural mesothelioma cell lines. We induced expression of ΔPDZ-Dvl-1 in LRK1A, REN, and H513 through retroviral transfection, using empty vector as a control. After selection, cells were plated in 0.35% soft agar and colonies scored after 28 days. Colony formation in LRK1A and REN transfected with ΔPDZ-Dvl-1 decreased substantially compared with control (P<0.01). H513 was unable to grow in soft agar. In addition, the in vivo growth of both LRK1A and REN s.c. tumors in athymic mice was inhibited significantly by transfection with a ΔPDZ-Dvl-1 mutant compared with control (P<0.05 and P<0.005, respectively; FIG. 8).

### Example 11

### Role of Dvl Activation in Non Small Cell Lung Cancer

We next examined the role of Dvl activation in non small-cell lung cancer (NSCLC). This example demonstrates that Dvl-3 is overexpressed in freshly resected NSCLC and established NSCLC cell lines. We example also provides additional evidence that Wnt signaling through canonical β-catenin pathways is due to upstream events, such as Dvl expression.

We analyzed Dvl expression and function in order to evaluate the function of wnt signaling in NSCLC. Eight NSCLC fresh tumors (four squamous cell and four adenocarcinomas) and their autologous matched normal lung tissue were obtained from patients undergoing resection of their tumors as part of their treatment for early stage I NSCLC. Patients had not received any prior treatment, e.g., chemotherapy. Western blot analysis of these samples showed that in 75% (three of four squamous cell carcinomas and three of four adenocarcinomas) of all cancer cells tested, Dvl-3 was overexpressed while the corresponding matched normal microdissected lung tissues failed to show expression of Dvl-3. Furthermore, five of six NSCLC tumors with Dvl-3 over-expression showed higher expression of Wnt-1 or Wnt-2 by western blot analysis. Expression of Dvl-1 or Dvl-2 was not detected.

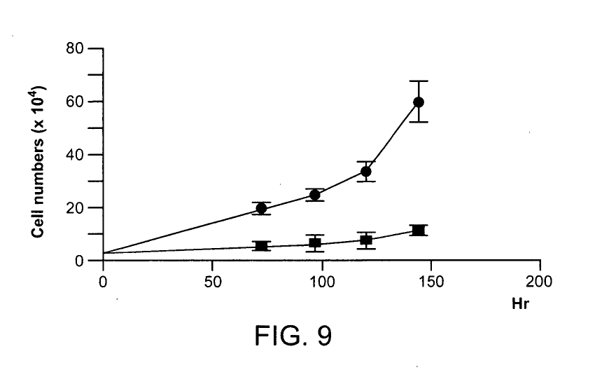

To further examine Dvl function, we synthesized small interfering RNA (siRNA) of Dvls that are capable of suppressing Dvl-1, -2, and -3. We tested the function of Dvl in the lung cancer cell line H1703 by treatment with Dvl siRNA and control siRNA. We chose H1703 because it expresses Dvl-3 and has been shown to exhibit Tcf-dependent transcriptional activity of β-catenin. After siRNA treatment, expression of dvl-3 was suppressed, while dvl-1 and -2 remained unexpressed. Of note, β-catenin expression decreased accordingly in treated cells, which was accompanied by a significant reduction in Tcf-dependent transcriptional activity (P<0.05). Lastly, siRNA of Dvls inhibited H1703 cell growth in 24-well plates significantly (P<0.05) (FIG. 9). In addition, colony formation in 100-mm dishes was also suppressed significantly (P<0.05). In other cell lines with lower levels of Dvl expression compared to that in H1703, such as A549 (a lung cancer cell line) and SW480 (a colon cancer cell line with aberrant activation in the Wnt signaling pathway due to APC mutation), cell growth was unaffected by the Dvl siRNA.

### Discussion

As noted above, little is known regarding the role that wnt ligand plays in human carcinogenesis. The data presented here demonstrate that wnt signals play a causal role in human cancer cells and thus are cancer therapeutic targets.

The data presented above demonstrate that both anti-wnt-1 and anti-wnt-2 antibodies can induce apoptosis in human cancer cells. Furthermore, our data indicates that the anti-tumor effect was due to the blockade of wnt signaling pathway. The apoptotic cell death induced by anti-Wnt antibody was not only correlated with the Wnt protein expression, but also consistent with the decreased dvl and cytosolic β catenin protein expression in the human tumor cells tested. Conversely, both Dvl and cytosolic β-catenin proteins remain the same level in normal cell lines after anti-Wnt antibody treatment. The antibodies showed no detectable effect on normal cell lines, suggesting that anti-Wnt-1 or anti-Wnt-2 antibody could specifically induce apoptosis in cancer cells, but not in normal cells. Given the possibility that polyclonal antibodies may generate non-specific effects, we used an anti-wnt-1 monoclonal antibody to further investigate the specificity of the effect of anti-wnt antibodies. The anti-wnt-1 monoclonal antibody was able to induce apoptosis in human cancer cell lines that over-express Wnt-1 protein, e.g., human lung cancer cell line H460 and human breast cancer cell line MCF-7. Similar to the results obtained from polyclonal antibody study, both dvl and cytosolic β catenin proteins were decreased after the anti-Wnt-1 monoclonal antibody treatment in these tumor cells. However, the anti-Wnt-1 monoclonal antibody showed much higher specificity than the anti-Wnt-1 polyclonal antibody, e.g., the anti-Wnt-1 monoclonal induces apoptosis only in the tumor cells that over-express Wnt-1 protein (H460 and MCF-7), and has no detectable effect in the tumor cells that express Wnt-2 protein; the anti-Wnt-1 polyclonal antibody induces apoptotic cell death in the tumor cells that over-express either Wnt-1 or Wnt-2. Taken together, these data indicate that the anti-Wnt antibody treatment can induce tumor-specific apoptosis and down-regulate the Wnt-dvl-β catenin signaling pathway in human cancer cells.

Through frizzled receptor and dishevelled protein, Wnt signal activates two distinct pathways: the canonical pathway (i.e., β catenin pathway) and the JNK pathway. Dishevelled protein has three highly conserved domains, DIX, PDZ, and DEP. Among them, the DIX and PDZ domains are necessary for the canonical signaling pathway while the DEP domain is important for the activation of JNK pathway. It has been suggested that the activation of JNK plays a critical role in initiating apoptosis (Wang et al., Mol Cell Biol, 19:5923-5929 (1999)). Recently, Chen et al. have demonstrated that Wnt-1 inhibits apoptosis by activating β catenin and TCF transcription (Chen et al., J Cell Biol, 152:87-96 (2001)). In this study, both over-expression of β-catenin and increased JNK activity were observed after anti-Wnt antibody treatment, suggesting that both the canonical pathway and the JNK pathway are involved in the apoptosis induced by anti-Wnt antibody. In addition, over-expression of Dvl in a normal mesothelial cell line down regulated JNK activities and the inhibition of Dvl by using Apigenin to block CK-1 activity increased JNK activity. Most likely, the activation of JNK after anti-Wnt antibody treatment is through Dvl.

Furthermore, siRNA-mediated inhibition of Dvl expression in NSCLC cells decreased β-catenin-mediated Tcf transcription, which further supports that Dvl overexpression is important to the canonical Wnt/B-catenin pathway in some lung cancer cells. Inhibition of Dvl also suppressed cell growth and colony formation in NSCLC cells, which indicates that aberrant upstream events in Wnt signaling is related to tumorigenesis in NSCLC.

Degradation of Dvl by siRNA resulted in growth suppression in H1703, but not in A549 cells. These are both squamous cell lung cancer cell lines, but H1703 has mutational inactivation of p53 whereas A549 has wild-type p53. The p53 status may therefore explain, at least in part, the differences in Dvl function between the two squamous cell lung cancer cell lines treated.

To further elucidate the mechanism through which anti-Wnt antibody induce apoptosis in human cancer cells, we have examined other possible components in the apoptotic pathway. For instance, releasing of Smac/Diablo into cytosol was detected in these tumor cells treated with wnt antibody. Smac/Diablo (second mitochondria-derived activator of caspase/direct IAP-binding protein with low pI) (Du et al., Cell, 102:33-42 (2000); Verhagen et al., Cell, 102:43-53 (2000)) functions by releasing the IAP-mediated caspase inhibition. Stimulation of apoptosis causes releasing of Smac/Diablo from the intermembrane space of mitochondria into the cytosol, together with cytochrome c. Cytochrome c directly activates Apaf-1 and caspase-9 and Smac/Diablo interacts with multiple LAPs to remove IAP-mediated inhibition of both initiator and effector caspases (Chai et al., Nature, 406:855-862 (2000); Srinivasula et al., J Biol Chem, 275:36152-36157 (2000)). Consistent with above results where caspase-3 activity increases in the cancer cells, but not in the normal cells, we found increase level of both Smac/Diablo and cytochrome c in the cytosol of the cancer cells after anti-Wnt antibody treatment, but not in that of the normal cells. Our results indicate that both Smac/Diablo and cytochrome c are likely involved in this anti-Wnt antibody induced apoptosis by removing survivin and/or other IAPs-mediated inhibition and direct activation of caspases, respectively.

The above findings suggest that wnt antibodies may not only induce directly apoptosis in cancer cell that overexpress wnt proteins, but also release potentially drug resistance by restoring normal apoptotic machinery back to these tumor cells. The basis for drug resistance in tumor cells is most likely the disruption of apoptosis. Over expression of Survivin, an inhibitor of apoptosis, is a common feature of most human cancers. It has been shown that targeting of survivin increases the sensitivity of tumor cells to cytotoxic drugs and that antisense survivin is sufficient to cause apoptosis in human mesothelioma cells. Moreover, a synergistic effect between antisense surviving and chemotherapy has also been reported.

We have shown that wnt antibody treatment dramatically decreases the protein expression level of Survivin. Taken together, Wnt antibody should potentiate and synergize the effect of standard chemotherapy in human cancer cells.

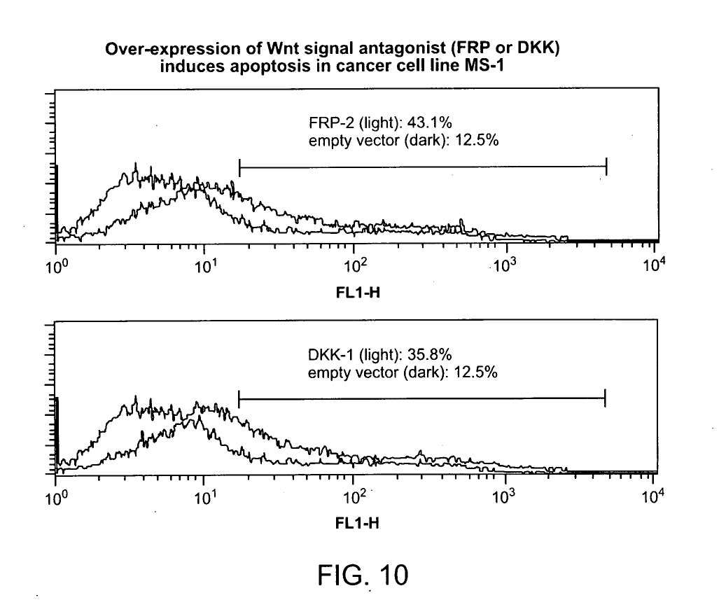

Other antagonists of Wnt signal or Frizzled receptor should also induce apoptosis through dishevelled. For instance, sFRPs function as soluble modulators of Wnt signaling by competing with the Frizzled receptors for the binding of secreted Wnt ligands (Melkonyan et al., Proc Natl Acad Sci USA, 94:13636-13641 (1997)). Specifically, sFRPs can either antagonize Wnt function by binding the protein and blocking access to its cell surface signaling receptor, or they can enhance Wnt activity by facilitating the presentation of ligand to the Frizzled receptors (Uthoff et al., Int J Oncol, 19:803-810 (2001)). Frizzled receptor antagonists (e.g., antibody specific for the extracellular domain or small molecule specific for the intracellular domain) should induce apoptosis in human cancer cells that overexpress wnt/frizzled proteins. Indeed, FIG. 10 shows that over-expression of Wnt signal antagonist, FRP or DKK, induces apoptosis in cancer cells. Thus, such antagonists can also be used to treat cancer, e.g., lung cancer, mesothelioma, breast cancer, colorectal cancer, cervical cancer, ovarian cancer, prostate cancer, pancreatic cancer, gastric cancer, esophageal cancer, head and neck cancer, hepatocellular carcinoma, melanoma, glioma, glioblastoma, leukemia, or lymphoma.

In summary, our results indicate that wnt monoclonal antibodies can induce tumor-specific apoptosis in human cancer cells, probably through both the canonical and the JNK pathways. Our data demonstrate that Wnt/Frizzled is a useful therapeutic targets for the treatment of cancer, and the results from xenograft mouse model implicate that Wnt monoclonal antibodies are good candidates of tumor-targeting cancer therapeutics.

### Example 12

### Analysis of Silencing Mechanisms of the Dachsous (ds) and Fat Genes and their Regulations of the Wnt-Frizzled Signaling Pathway in Human Cancers

Little is known regarding modification machinery of Wnt-Fz signaling in cancers. Dachsous (Ds) and Fat proteins are two cadherin superfamily members (Mahoney, et al., Cell 67: 853-868, 1991; Clark, H. F., et al., Genes Dev, 9: 1530-1542, 1995). They have been shown to participate in Fz signaling in Drosophila development (Yang, et al., Cell, 108: 675-688, 2002). There is no report on the role of Ds and Fat in cancers.

In this example, we show that in fresh human cancer tissues (including lung cancer and mesothelioma) and human cell lines (including breast cancer, colon cancer, lung cancer and mesothelioma) Fat expression was upregulated and Ds expression was downregulated. We also identified aberrant methylation in the CpG island of Ds promoter region that correlated with Ds transcription silencing in human cancers. In addition, we found that Fz activity was correlated with upregulation of Fat and downregulation of Ds. Restoration of Ds and blockage of Fat could modulate activity of the Fz signaling pathway and suppress cancer cell growth.

This invention is of great help in therapeutic strategies for the treatment of human cancers. For example, using the methods described above, Fat activity can be blocked. Such methods include, for example, antisense oligonucleotides or small chemical molecules to block Fat transcript and/or its activity. Alternatively, Ds activity can be restored using known gene therapy methods. In addition, this invention can be used as a diagnostic tool. Methylation is one of early events in cancer formation. Methylation detection in the CpG island of Ds promoter region using well known techniques, for example, methylation-specific PCR can be used on early cancer diagnosis.

### Example 13

### Genetic Alterations in Frizzled (fz) Genes and LRP (LDL-Related Protein) Genes and Targeting Mutant and/or Truncated Forms of these Receptors Using Different Methods in Cancers

LRPs (LDL-related protein; LRP-1 to 5) are co-receptors for Wnt ligands. It has been shown that Wnt, Fz and LRP proteins often have high level expression in a number of cancers, including breast cancer, colon cancer, lung cancer etc. (Liu, et al., Cancer Res, 60: 1961-1967, 2000; Laurencot, et al., Int J Cancer, 72: 1021-1026, 1997; Berger, W., et al., Int J Cancer, 88: 293-300, 2000; Schneider, et al., Breast Cancer Res, 3: 183-191, 2001; Schneider, et al., Anticancer Res, 20: 4373-4377, 2000). In addition, this signaling is thought to turn on downstream transcriptional activity constitutively in cancers. However, mechanisms in this constitutive-on signaling in cancers still remain unsolved.

In this example, we show that genetic alterations in frizzled (fz) genes and/or LRP (LDL-related protein) genes result in mutant and/or truncated forms of all Fz receptors and/or LRP co-receptors (extracellular, transmembrane, and/or intracellular domains) in cancers. The cancer types that we tested include breast cancer, colon cancer, prostate cancer, lung cancer, mesothelioma, and sarcoma. The genetic alterations mentioned above include chromosomal deletion (homozygous or heterozygous), chromosomal translocation, chromosomal breaks, chromosomal inversions, internal small deletions, insertions, and point mutations. These mutant and/or truncated forms of Fz receptors and/or LRP co-receptors result in constitutive signaling regardless presence of Wnt ligands, which in turn result in constitutive downstream transcriptional activities in cancers. In contrast, there are no mutant forms of Fz receptors and/or LRP co-receptors in normal cells/tissues.

This invention demonstrates that mutant and/or truncated forms of Fz receptors and/or LRP co-receptors for the Wnt signaling pathway are cancer specific. They have very strong potential to be used as targets for developing therapeutic drugs (e.g., small molecules, chemical compounds, antibodies, antisense-oligos or RNAi as discussed above). These drugs are able to target cancers only, but not normal cells. Thus, this invention will be of great help in therapeutic strategies for treatment of a number of cancers as noted above, including colon cancer, breast cancer, lung cancer, e.g., NSCLC, mesothelioma and sarcoma, and the like.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1.-35. (canceled)

36. A method of reducing the proliferation of a human cancer cell that overexpresses a Wnt1 protein in a patient comprising:
administering to said patient an antibody specific for the Wnt1 protein, wherein said administering reduces the proliferation of said human cancer cell.

37. The method of claim 36, wherein the antibody is a humanized antibody.

38. The method of claim 36, wherein the antibody is a single chain Fv fragment (scFv).

39. The method of claim 36, wherein the cancer cell is a breast cancer cell.

40. The method of claim 36, wherein the cancer cell is a lung cancer cell.

41. The method of claim 36, further comprising administering a second cancer therapeutic agent to said patient.

42. The method of claim 41, wherein said second therapeutic agent is an agent for chemotherapy.

43. A method of reducing the proliferation of a human cancer cell that overexpresses a Wnt2 protein in a patient comprising:
administering to said patient an antibody specific for the Wnt2 protein, wherein said administering reduces the proliferation of said human cancer cell.

44. The method of claim 43, wherein the antibody is a humanized antibody.

45. The method of claim 43, wherein the antibody is a single chain Fv fragment (scFv).

46. The method of claim 43, wherein the antibody competes for binding to a Wnt2 protein with an antibody that comprises
(i) a V_{L}CDR1 comprising an amino acid sequence of a V_{L}CDR1 of FIG. 7;
(ii) a V_{L}CDR2 comprising an amino acid sequence of a V_{L}CDR2 of FIG. 7;
(iii) a V_{L}CDR3 comprising an amino acid sequence of a V_{L}CDR3 of FIG. 7;
(iv) a V_{H}CDR1 comprising an amino acid sequence of a V_{H}CDR1 of FIG. 7;
(v) a V_{H}CDR2 comprising an amino acid sequence of a V_{H}CDR2 of FIG. 7; and
(vi) a V_{H}CDR3 comprising an amino acid sequence of a V_{H}CDR3 of FIG. 7.

47. The method of claim 43, wherein the cancer cell is a breast cancer cell.

48. The method of claim 43, wherein the cancer cell is a lung cancer cell.

49. The method of claim 43, further comprising administering a second cancer therapeutic agent to said patient.

50. The method of claim 49, wherein said second therapeutic agent is an agent for chemotherapy.
